(19) United States
(12) Patent Application Publication
(10) Pub. No.: US 2015/0275306 A1
(43) Pub. Date: Oct. 1, 2015

(54) METHODS AND MEANS FOR PREDICTING RESISTANCE TO ANTI-CANCER TREATMENT

(71) Applicants: Stichting Het Nederlands Kanker Instituut Antoni van Leeuwenhoek Ziekenhuis, Amsterdam (NL); AGENDIA N.V., Amsterdam (NL)

(72) Inventors: Rene Bernards, Amsterdam (NL); Paul Roepman, Amsterdam (NL); Sidong Huang, Amsterdam (NL)

(21) Appl. No.: 14/434,474

(22) PCT Filed: Oct. 10, 2013
(86) PCT No.: PCT/NL2013/050720

§ 371 Date: Apr. 9, 2015

(30) Foreign Application Priority Data
Oct. 10, 2012 (EP) 12188037.1

(51) Int. Cl. C12Q 1/68; G01N 33/574

(52) U.S. Cl. CPC C12Q 1/6886; G01N 33/57484; C12Q 2600/158; C12Q 2600/16; G01N 2800/52

(57) ABSTRACT

The present invention relates to methods of typing a sample from an individual suffering from cancer. The invention further relates to methods for assigning treatment to an individual suffering from cancer, comprising typing a sample from an individual suffering from cancer according to the methods of the invention.

### FIELD

The invention relates to the field of cancer diagnostics, more specifically to new methods and means for typing a sample from an individual suffering from cancer. The methods and means of the invention will assist in the prediction of whether a cancer is resistant to anti-cancer treatment. In addition, the invention provides methods and means for assigning treatment to an individual suffering from a cancer that has been typed as being or becoming resistant to anti-cancer therapy.

### INTRODUCTION

Cancer therapy is often hampered by the rapid emergence of drug resistance. This is not only true for the conventional chemotherapies, but also for the new generation of drugs targeting those components that are mutated or deregulated in cancer cells. For example, treatment of metastatic non-small-cell lung cancers (NSCLCs) harboring activating mutations in the gene encoding the Epidermal Growth Factor Receptor (EGFR) leads to dramatic initial responses, resulting in significant increase in progression free survival. However, such responses are often short-lived resulting in much less impressive patient benefit in terms of overall survival (Maemondo et al., 2010. N Engl J Med 362: 2380-2388). This lack of overall survival benefit is the consequence of the rapid emergence of drug resistant variants. Development of resistance to targeted therapies is a quite general phenomenon and is also seen in BCR-ABL translocated chronic myelogenous leukemias (CML) treated with imatinib (Gorre et al., 2001. Science 293: 876-880; Shah et al., 2002. Cancer Cell 2: 117-125), BRAF mutant melanomas treated with the BRAF inhibitor vemurafenib (Chapman et al., 2011. N Engl J Med 364: 2507-2516) and EML4-ALK translocated NSCLCs treated with the ALK inhibitor crizotinib (Kwak et al., 2010. N Engl J Med 363: 1693-1703).

About half of the resistance seen in EGFR mutant NSCLCs treated with EGFR inhibitors can be explained by secondary mutations in the EGFR gene itself (Sequist et al., 2011. Science Transl Med 3: 75ra26). The T790M “gatekeeper” mutation in EGFR is critical for binding of competitive inhibitors to the ATP-binding pocket (Daub et al., 2004. Nat Rev Drug Discov 3: 1001-1010; Yun et al., 2008. Proc Natl Acad Sci USA 105: 2070-2075.), allowing continued proliferation in the presence of the drug. Similar gatekeeper mutations have been found in BCR-ABL positive CMLs treated with imatinib (Science 293: 876-880; Shah et al., 2002. Cancer Cell 2: 117-125) and in EML4-ALK mutant NSCLCs treated with crizotinib (Choi et al., 2010. N Engl J Med 363: 1734-1739; Katayama et al., 2011. Proc Natl Acad Sci USA 108: 7535-7540). Resistance to targeted therapies that does not involve secondary mutations in the drug target itself is often caused by mutations in the components of the signaling pathway downstream of the target. Thus, primary resistance to EGFR-targeted therapy in colon cancer is associated with mutations in KRAS (Amado et al., 2008. J Clin Oncol 26: 1626-1634; Karapetis et al., 2008. N Engl J Med 359: 1757-1765). Similarly, acquired resistance to targeted therapies often selects for mutations in components of the signaling pathway downstream of the drug target. For example, resistance to BRAF inhibition in melanoma can result from an activating mutation in the MEK1 kinase that was not detectable in the primary cancer (Wagle et al., 2011. J Clin Oncol 29: 3085-3096). Moreover, mutations in PIK3CA, the catalytic subunit of PI-3kinase, are seen in some 5% of NSCLCs that develop resistance to EGFR targeted therapies (Sequist et al., 2011. Science Transl Med 3: 75ra26). Alternatively, resistance can result from activation of a parallel pathway or in genes that feed into the downstream signaling of the drug target. Thus, amplification of the MET oncogene is found in EGFR drug resistant NSCLC (Sequist et al., 2011. Science Transl Med 3: 75ra26) and over-expression of COT, leading to activation of MEK, can be a causal agent in BRAF resistance in melanoma (Johannessen et al., 2010. Nature 468: 968-972.). At present, some 30% of the resistance to EGFR targeted therapies in NSCLCs cannot be explained by any of the mechanisms described above (Sequist et al., 2011. Science Transl Med 3: 75ra26). Hence, there is a clear need to identify novel mechanisms that are involved in mediating drug resistance, and which provide novel entry points to overcome the resistance to cancer therapy.

### SUMMARY OF THE INVENTION

The invention provides a method of typing a sample from an individual suffering from cancer, the method comprising determining a level of expression for a set of at least 5 genes that are selected from Table 1 in a relevant sample from the individual, whereby the sample comprises expression products from a cancer cell of the patient, comparing said determined level of expression of the set of genes to the level of expression of the set of genes in a reference sample or reference population, and typing said sample based on the comparison of the determined levels of expression.

The present inventors have used a large-scale loss-of-function genetic screen to identify genes whose suppression can confer resistance to crizotinib in a NSCLC cell line harboring an EML4-ALK translocation. A key component of the transcriptional MEDIATOR complex, MED12, was surprisingly identified as a determinant of crizotinib response in NSCLC. It was further established that suppression of MED12 also confers resistance to a range of anti-cancer treatment, including chemotherapy, in a wide range of cancers, including colon cancer, melanoma, breast and liver cancer.

The levels of expression of the genes listed in Table 1 were found to be indicative of the activity of MED12.

In a preferred method according to the invention, the set of genes comprises at least ten genes that are selected from Table 1. A further preferred set of genes according to the invention comprises forty-one genes that are selected from Table 1 and which are rank-ordered 1-41; more preferred at least forty-six genes that are selected from Table 1 and that are rank-ordered 1-46.

In a preferred method according to the invention, the sample comprises RNA expression products and the level of expression for a set of genes that are selected from Table 1 is determined by determining the level of expression of RNA molecules that are encoded by the set of genes.

Typical cancers for typing according to the methods of the invention include breast cancer (e.g., BRCA-1 deficient, stage-III HER2-negative, luminal type, basal type, ERBB2 type, ER/PR positive, HER2 positive, ductal carcinoma, lobular carcinoma), ovarian cancer (e.g., BRCA-1 deficient, epithelial ovarian cancer), lung cancer (e.g., non-small-cell lung cancer or small cell lung cancer, metastatic non-small cell lung cancer), liver cancer (e.g., hepatocellular carcinoma), head and neck cancer (e.g., metastatic squamous cell carcinoma of the head and neck (SCCHN), squamous cell carcinoma, laryngeal cancer, hypopharyngeal cancer, oropharyngeal cancer, and oral cavity cancer), bladder cancer (e.g., transitional cell carcinoma of the bladder), and colorectal cancer (e.g., advanced (non-resectable locally advanced or metastatic) colorectal cancer). Other cancers for which the methods and compositions of the invention may provide predictive treatment include cervical cancer (e.g., recurrent and stage IVB), mesothelioma, solid tumors (e.g., advanced solid tumors), renal cell carcinoma (e.g., advanced renal cell carcinoma), stomach cancer, sarcoma, prostate cancer (e.g., hormone refractory prostate cancer), melanoma, thyroid cancer (e.g., papillary thyroid cancer), brain cancer, adenocarcinoma, subependymal giant cell astrocytoma, endometrial cancer, glioma, glioblastoma, and other tumors or cancers that have metastasized to the brain, esophageal cancer, neuroblastoma, hematological cancers, and lymphoma. Said cancer is preferably selected from colorectal cancer, lung cancer, liver cancer, prostate cancer and breast cancer.

Said typing is preferably used to predict whether the individual has a high risk of being or becoming resistant to anti-cancer treatment (MED12-knock down like), or a low risk of being or becoming resistant to anti-cancer treatment (MED12wild type). For this, the level of expression of the set of genes selected from Table 1 is determined in a relevant sample from the individual, whereby an alteration in the level of expression, when compared to the level of expression of the set of genes in a relevant reference sample or reference population.

The methods of the invention preferably comprise determining a similarity value between the determined level of expression of the set of genes in an individual suffering from cancer and the level of expression of said set of genes in a relevant reference sample or reference population. The individual is classified as having a high risk of being or becoming resistant to anti-cancer treatment if said similarity value is below a first similarity threshold value, and classifying said individual as having a low risk of being or becoming resistant to anti-cancer treatment if said similarity value exceeds said first similarity threshold value.

Said anti-cancer treatment is preferably selected from an alkylating agent such as nitrogen mustard, e.g. cyclophosphamide, mechlorethamine or mustine, uramustine or uracil mustard, melphalan, chlorambucil, ifosfamide; a nitrosourea such as carmustine, lomustine, streptozocin; an alkyl sulfonate such as busulfan, an ethylenime such as thiotepa and analogues thereof, a hydrazine/triazine such as dacarbazine, altretamine, mitozolomide, temozolomide, altretamine, procarbazine, dacarbazine and temozolomide, which are capable of causing DNA damage; an intercalating agent such as a platinum agent like cisplatin, carboplatin, nedaplatin, oxaliplatin and satraplatin; an antibiotic such as an anthracycline such as doxorubicin, daunorubicin, epirubicin and idarubicin; mitomycin-C, dactinomycin, bleomycin, adriamycin, mithramycin; an antimetabolite such as capecitabine and 5-fluorouracil, gemcitabine, a folate analogue such as methotrexate, hydroxyurea, mercaptopurine, thioguanine; a mitostatic agent such as eribulin, ixabepilone, irinotecan, vincristine, mitoxantrone, vinorelbine and a taxane such as paclitaxel and docetaxel; a receptor tyrosine kinase inhibitor such as gefitinib, erlotinib, EKB-569, lapatinib, CI-1033, cetuximab, panitumumab, PKI-166, AEE788, sunitinib, sorafenib, dasatinib, nilotinib, pazopanib, vandetaniv, cediranib, afatinib, motesanib, CUDC-101, and imatinib mesylate; a MEK inhibitor including CKI-27, RO-4987655, RO-5126766, PD-0325901, WX-554, AZD-8330, G-573, RG-7167, SF-2626, GDC-0623, RO-5068760, and AD-GL0001; a B-RAF inhibitor including CEP-32496, vemurafenib, GSK-2118436, ARQ-736, RG-7256, XL-281, DCC-2036, GDC-0879, AZ628, an antibody fragment EphB4/Raf inhibitor; a serine/threonine kinase receptor inhibitor, including an Alk-1 inhibitor such as crizotinib, ASP-3026, LDK378, AF802, and CEP37440, and combinations thereof.

Said anti-cancer treatment is more preferably selected from a platinum agent like cisplatin, carboplatin, oxaliplatin and satraplatin; taxane including paclitaxel and docetaxel, doxorubicin, daunorubicin, epirubicin, cyclophosphamide, fluorouracil, gemcitabine, eribulin, ixabepilone, methotrexate, mutamycin, mitoxantrone, vinorelbine, thiotepa, vincristine, capecitabine, a receptor tyrosine kinase inhibitor and/or irinotecan.

A preferred method according to the invention further comprises determining a strategy for treatment of the patient.

Downmodulation of MED12 was found to result in increased cell surface expression of the TGFbeta receptor and to enhance TGFbeta receptor-mediated signaling activity, resulting in a morphological alteration of the cells, resembling epithelial-mesenchymal transition. The enhanced TGFbeta receptor-mediated signaling activity was found to induce resistance to anti-cancer treatment. Consequently, it was shown that inhibition or downregulation of TGFbeta receptor signaling is able to overcome resistance to anti-cancer treatment.

Therefore, in a preferred method according to the invention said strategy for treatment of a patient that was typed or classified as having a high risk of being or becoming resistant to anti-cancer treatment comprises anti-TGFbeta treatment, either alone or, preferably, in combination with said anti-cancer therapy.

Therefore, the invention further provides a method for assigning treatment to an individual suffering from cancer, comprising (a) typing a relevant sample from the patient according to the method of the invention; (b) classifying said sample as having a high risk of being or becoming resistant to anti-cancer treatment or as having a low risk of being or becoming resistant to anti-cancer treatment; and (c) assigning anti-TGFbeta treatment to an individual of which the sample is classified as having a high risk of being or becoming resistant to anti-cancer treatment. It is preferred that the anti-TGFbeta treatment is combined with said anti-cancer treatment.

A preferred anti-TGFbeta treatment comprises the administration of LY2157299, which is a potent inhibitor of TGFbeta receptor Type I and II (transforming growth factor 6 receptor I and II) with IC50 of 86 nM and 2 nM, respectively.

### FIGURE LEGENDS

FIG. 1 A genome-wide RNAi screen identifies MED12 as a critical determinant of drug response to tyrosine kinase inhibitors in NSCLCs

(A) Schematic outline of the crizotinib resistance barcode screen performed in H3122 cells. NKI human shRNA library polyclonal virus was produced to infect H3122 cells, which were then left untreated (control) or treated with 300 nM crizotinib for 14 or 28 days, respectively. After selection, shRNA inserts from both populations were recovered, labeled and hybridized to DNA oligonucleotide barcode arrays.

(B) Analysis of the relative abundance of the recovered shRNA cassettes from crizotinib barcode experiment. Averaged data from three independent experiments were normalized and 2 log transformed. M- and A-values reflect relative enrichment and hybridization signal intensity, respectively. Among the 43 top shRNA candidates (M>2 and A>7), two independent shMED12 vectors (arrows) were identified.

(C to E) Three independent shRNAs targeting MED12 confer resistance to ALK inhibitors. C) The functional phenotypes of non-overlapping retroviral shMED12 vectors (#1-3) in H3122 cells are indicated by colony formation assay in 300 nM crizotinib or 2.5 nM NVP-TAE684. The pRS vector was used as a control. The cells were fixed, stained and photographed after 14 (untreated) or 28 days (treated). D) The level of MED12 knockdown by each of the shRNAs was measured by examining the MED12 mRNA levels by qRT-PCR. Error bars denote standard deviation (SD). E) The level of knockdown of MED12 protein was measured by western blotting.

(F to H) Suppression of MED12 also confers to EGFR inhibitors. F) Colony formation assay of PC9 cells expressing pLKO control or independent lentiviral shMED12 vectors (#4 and #5) were cultured in 50 nM gefitinib or 50 nM erlotinib. The cells were fixed, stained and photographed after 10 (untreated) or 28 days (treated). G) The level of knockdown of MED12 by each of the shRNAs was measured by examining the MED12 mRNA levels by qRT-PCR. Error bars denote SD. H) The level of knockdown of MED12 protein was measured by western blotting.

FIG. 2. MED12 suppression leads to MEK/ERK activation and confers multi-drug resistance in different cancer types

(A and B) Downregulation of MED12 results in elevated level of phosphorylated MEK (p-MEK) and phosphorylated ERK (p-ERK). A) MED12KD H3122 cells have higher p-MEK and p-ERK levels. H3122 cells expressing pLKO control or shMED12 vectors were grown in the absence or presence 20 nM NVP-TAE684 for 6 hours and the cell lysates were harvested for western blotting analysis. B) Elevated p-MEK and p-ERK levels in MED12KD PC9 cells. PC9 cells expressing pLKO control or shMED12 vectors were grown in the absence or presence of 25 nM gefitinib for 6 hours and the cell lysates were harvested for western blotting analysis.

(C and D) MED12 knockdown confers resistance to BRAF and MEK inhibitors in melanoma cells. C) BRAFV600E A375 cells expressing pLKO control or shMED12 vectors were cultured in the absence or presence of 2.5 μM PLX4032 or 0.5 μM AZD6244. The cells were fixed, stained and photographed after 10 (untreated) or 28 days (treated). D) MED12 suppression results in elevated level of p-ERK in melanoma cells. A375 cells expressing pLKO control or shMED12 vectors were grown in the absence or presence of 1 μM PLX4032 or 0.5 μM AZD6244 for 6 hours and the cell lysates were harvested for western blotting analysis.

E-F) MED12 knockdown confers resistance to MEK inhibitor in colorectal cancer cells. E) KRASV12 SK-CO-1 cells expressing pLKO control or shMED12 vectors were cultured in the absence or presence of 0.5 μM AZD6244. The cells were fixed, stained and photographed after 14 (untreated) or 28 days (treated). F) MED12 suppression results in elevated level of p-ERK in colorectal cancer cells. SK-CO-1 cells expressing pLKO control or shMED12 vectors were grown in the absence or presence of 1 μM AZD6244 for 6 hours and the cell lysates were harvested for western blotting analysis.

(G-H) Knockdown of MED12 confers resistance to multi-kinase inhibitor sorafenib in HCC Huh-7 cells. G) Colony formation assay of Huh-7 cells expressing pLKO control or shMED12 vectors (#4 and #5) were cultured in 2 μM sorafenib. The cells were fixed, stained and photographed after 14 (untreated) or 21 days (treated). H) MED12 suppression results in elevated level of p-ERK in HCC cells. Huh-7 cells expressing pLKO control or shMED12 vectors were grown in the absence or presence of 4 μM sorafenib for 6 hours and the cell lysates were harvested for western blotting analysis.

FIG. 3 MED12 suppression confers multi-drug resistance in cell lines of different cancer types

(A) Downregulation of MED12 retains elevated level of p-MEK in the presence of MEK inhibitor. A375 cells expressing pLKO control or shMED12 vectors were grown in the absence or presence of 25 nM MEK inhibitor PD0325901 for 6 hours and the cell lysates were harvested for western blotting analysis.

(B-C) knockdown of MED12 confers resistance to BRAF and MEK inhibitors in melanoma SK-MEL-28 (BRAFV600E) cells. B) Colony formation assay of SK-MEL-28 cells expressing pLKO control or shMED12 vectors (#4 and #5) were cultured in 5 μM PLX4032 or 1 μM AZD6244. The cells were fixed, stained and photographed after 14 (untreated) or 28 days (treated). C) The level of knockdown of MED12 by each of the shRNAs was measured by examining the MED12 mRNA levels by qRT-PCR. Error bars denote SD.

(D-E) knockdown of MED12 confers resistance to BRAF and MEK inhibitors in CRC SW1417 (BRAFV600E) cells. D) Colony formation assay of SW1417 cells expressing pLKO control or shMED12 vectors (#4 and #5) were cultured in 2 μM PLX4032 or 150 nM AZD6244. The cells were fixed, stained and photographed after 14 (untreated) or 28 days (treated). E) The level of knockdown of MED12 by each of the shRNAs was measured by examining the MED12 mRNA levels by qRT-PCR. Error bars denote SD.

(F-G) knockdown of MED12 also confers resistance to chemotherapy drugs. F) Colony formation assay of H3122 cells expressing pLKO control or shMED12 vectors (#4 and #5) were cultured in 2 μM cisplatin or 2.5 μM 5-FU. The cells were fixed, stained and photographed after 12 (untreated) or 18 days (treated). G) Colony formation assay of PC9 cells expressing pLKO control or shMED12 vectors (#4 and #5) were cultured in 0.37 or 1.1 μM cisplatin. The cells were fixed, stained and photographed after 10 (untreated) or 21 days (treated).

FIG. 4 TGFbeta signaling is required for the drug resistance driven by MED12 suppression

A) Schematic outline of the “drop out” RNAi screen for kinases whose inhibition restores sensitivity to crizotinib in MED12KD cells. Human TRC kinome shRNA library polyclonal virus was produced to infect H3122 cells stably expressing shMED12#3, which were then left untreated (control) or treated with 300 nM crizotinib for 10 days. After selection, shRNA inserts from both populations were recovered by PCR and identified by next generation sequencing.

B) Representation of the relative abundance of the shRNA bar code sequences from the shRNA screen experiment depicted in panel A. The y-axis is enrichment (relative abundance of crizotinib treated/untreated) and x-axis is the intensity (average sequence reads in untreated sample) of each shRNA. Among the 51 top shRNA candidates (more than 2.5-fold depleted by crizotinib treatment and more than 200 reads in untreated as indicated by the dash lines), two independent shTGFbeta R2 vectors (arrows) were identified.

C) Suppression of TGFbeta R2 restores the crizotinib sensitivity in MED12KD cells. Using lentiviral infection, pLKO control or two independent shTGFbeta R2 vectors were introduced into H3122 control or MED12KD cells. After this, cells were cultured in the absence or presence of 300 nM crizotinib. The cells were fixed, stained and photographed after 14 (untreated) or 21 days (treated).

D) The level of knockdown of TGFbeta R2 by each of the shRNAs was measured by examining the MED12 mRNA levels by qRT-PCR. Error bars denote SD.

E-F) Activation of TGFbeta signaling by TGFbeta R2 overexpression was sufficient to confer resistance to crizotinib in H3122 cells. E) H3122 cells expressing pQXCIP-GFP control or pQXCIP-TGFbeta R2-HA were cultured in the absence or presence of 300 nM crizotinib. The cells were fixed, stained and photographed after 14 (untreated) or 21 days (treated). F) Western blotting analysis showing that TGFbeta R2 overexpression resulted in elevated levels of phosphorylated SMAD2 (p-SAMD2) and p-ERK.

G) Activation of TGFbeta signaling by recombinant TGFbeta treatment also leads to resistance to crizotinib in H3122 cells in a TGFbeta-dosage dependent manner.

FIG. 5 MED12 suppresses TGF-beta signaling by negatively regulating TGF-beta receptor signaling in additional cell line models

(A-F) Downregulation of MED12 leads to induction of a panel of TGFbeta target genes and EMT marker genes. mRNA expression analysis by qRT-PCR of TGFbeta target genes ANGPTL4 (A), TAGLN (B), CYR61 (C) and CTGF (D) and EMT marker genes VIM (E) and CDH2 (F) in A375, SK-CO-1 and Huh-7 cells expressing pLKO controls or shMED12. Cells were cultured in normal condition without TGFbeta stimulation. Error bars denote SD.

(G) mRNA levels of TGFbeta 1 in A375, H3122 and PC9 cells expressing pLKO control or shMED12 were documented by qRT-PCR. Error bars denote SD.

(H) 125I labeled TGFbeta crosslinking assays showing the strong increase of cell surface TGFbeta R2 in H3122 cells expressing shMED12 but not pLKO. As a control, 125I-BMP9 affinity-labeling experiments performed in the same cells showed no significant change of cell surface BMP receptors.

(I-J) MED12 suppression results in strong induction of TGFbeta R2 protein and SMAD2 phosphorylation. Western blot analysis of A375 (I) and SK-CO-1 (J) cells expressing pLKO control or shMED12 vectors. Alpha-TUBULIN was used as a loading control.

K) MED12 localizes to both nucleus and cytoplasm. Western blotting analysis of the nuclear and cytoplasmic fractions prepared from H3122 cells expressing control vector or shMED12 with or without 16 hours of 300 nM crizotinib treatment. Lamin A/C and SP1 were used as marker controls for nuclear fractions, while alpha-TUBULIN and HSP90 were used as controls for cyctoplasmic fractions.

L) Western blotting showing that MED12 knockdown leads to induction of mesenchymal markers Vimentin and N-cadherin in Huh-7 cells.

M) Western blotting showing that recombinant TGFbeta treatment in Huh7 cells leads to increased levels of Vimentin, N-cadherin and p-ERK. Huh7 cells were first cultured in the presence of 50 picoM of TGFbeta for 6 days, and were then grown in the absence or presence of 4 microM sorafenib for 6 hours and the cell lysates were harvested for western blotting analysis.

FIG. 6 MED12 can be found in the direct proximity of endogenous TGFbeta R2 and it predominantly associates with immature forms of TGFbeta R2

A) MED12KD PC9 cells reconstituted with Flag-Med12 expressed at comparable levels as endogenous MED12 in parental PC9 cells. These cells were used for the Proximity Ligation Assay (PLA) described below.

B) Confocal images of the PLA experiments showing significant staining, which indicates direct interaction of MED12 and TGFbeta R2, in MED12KD PC9 cells reconstituted with Flag-Med12 (right panel), but not in the control cells lacking expressing of Flag-Med12 (left panel). Same cells were also stained with DAPI. Mouse anti-Flag and rabbit anti-TGFbeta R2 were used for the assay.

C) MED12 predominantly associates with immature forms of TGFbeta R2 that are not fully glycosylated. Western blotting analysis of co-immunoprecipitation experiments using antibodies against HA tag and MED12 on Phoenix cells co-transfected with HA-TGFbeta R2 and MED12 (5:1). The immunoprecipitates were incubated with Endo H or PNGase F enzymes, before loaded in the SDS-PAGE for the western blotting analysis. Antibodies against TGFbeta R2 and HA were used to detect TGFbeta R2 protein and showed identical results

FIG. 7 TGFbeta activation is sufficient to confer multi targeted drug resistance in different cancer types

(A-D) Overexpression of TGFbeta R2 was sufficient to induce expressions of TGFbeta target genes and EMT marker genes. mRNA expression analysis by qRT-PCR of TGFbeta target genes TAGLN (A) and ANGPTL4 (B), and EMT marker genes VIM (C) and CDH2 (D) in H3122 cells expressing pQXCIP-GFP control or pQXCIP-TGFbeta R2-HA. Cells were cultured in normal condition without TGFbeta stimulation. Error bars denote SD.

(E-G) Recombinant TGFbeta treatment leads to resistance to gefitinib (50 nM) in PC9 cells (E), AZD6244 (0.5 μM) in SK-CO-1 cells (F), PLX4032 (2.5 μM) and AZD6244 (0.5 μM) in A375 cells (G), cisplatin (2 μM) in H3122 (H) and (0.7 μM) PC9 cells (I) in a TGFbeta-dosage dependent manner.

FIG. 8 MED12KD signature predicts drug responses to cancer therapies.

A) Kaplan-Meier analysis of disease specific survival (DSS) for the cohort of 117 CRC cancers with MED12KD like gene signature. Patients with MED12KD like cancers treated with chemotherapy did not show significant DSS than untreated patients (red line, chemotherapy; black line, no treatment; HR=0.87; p=0.66). HR=hazard ratio; p=p-value.

B) Kaplan-Meier analysis of disease specific survival (DSS) for the cohort of 153 CRC cancers with MED12 wt like gene signature. Patients with MED12 wt like cancers treated with chemotherapy showed significant DSS than untreated patients (red line, chemotherapy; black line, no treatment; HR=0.33; p=0.00038). HR=hazard ratio; p=p-value.

C) MED12KD signature predicts drug responses to MEK inhibitors in 152 cell lines of different cancer types harboring the matching RAS or RAF mutations. High expression of subsets of genes upregulated in the MED12KD signature is significantly associated with higher IC50s for all four MEK inhibitors in (AZD6244, p=0.009; CI-1040, p=0.004; PD-0325901, p=0.007; RDEA119, p=0.013). Across these gene sets, each cell line was scored for the percentage of times it had high expression of the gene as well as being resistant to the inhibitor. The heatmap in the left panel of this figure depicts this percentage for each MEK inhibitor. The cell lines are sorted using hierarchical clustering for visualization. The middle and right panel depict the tissue type of the cell lines and their RAS/RAF mutation status.

FIG. 9 TGFbetaR inhibitor and TKIs synergize to suppress proliferation of MED12KD NSCLC cells.

A) Combination of TGFbeta R and ALK inhibitors synergistically inhibits growth of MED12KD NSCLC cells harboring EML4-ALK translocation. H3122 cells expressing pRS control or shMED12 vectors were cultured in the absence and the presence of 1 μM LY2157299, 300 nM crizotinib, or the combination of 1 μM LY2157299 and 300 nM crizotinib. The cells were fixed, stained and photographed after 14 (untreated and LY2157299 alone) or 28 days (crizotinib alone and LY2157299 plus crizotinib).

B) Combination of TGFbetaR and EGFR inhibitors synergistically inhibits growth of MED12KD NSCLC cells harboring EGFR activating mutation. PC9 cells expressing pLKO control or shMED12 vectors were cultured in the absence and the presence of 1 μM LY2157299, 100 nM gefitinib, or the combination of 1 μM LY2157299 and 100 nM gefitinib. The cells were fixed, stained and photographed after 10 (untreated and LY2157299 alone) or 28 days (gefitinib alone and LY2157299 plus gefitinib).

C-D) Combination of LY2157299 with crizotinib or gefitinib suppressed the ERK activation driven by MED12KD in both H3122 and PC9 cells. C) H3122 cells were grown in the absence or presence of 20 μM NVP-TAE684, 5 μM LY2157299 or the combination of 20 μM NVPTAE684 and 5 μM LY2157299 for 6 hours and the cell lysates were harvested for western blotting analysis. D) PC9 cells were grown in the absence or presence of 25 nM gefitinib, 5 μM LY2157299 or the combination of 25 nM gefitinib and 5 μM LY2157299 for 6 hours and the cell lysates were harvested for western blotting analysis.

FIG. 10 MED12KD signature is both prognostic and predictive

A) Kaplan-Meier analysis of disease specific survival (DSS) for the cohort of 231 CRC. MED12KD gene signature was used to hierarchically cluster the 231 CRC cancers into a cluster with poor DSS (cluster 1, black line, higher overall expression of genes upregulated in MED12KD signature) and one with significantly better DSS (cluster 2, red line, lower overall expression of genes upregulated in MED12KD signature).

B) IC50 values for AZD6244 and expression levels for ZBED2 across the 152 RAF/RAS mutated lines. The top panel represents a histogram of IC50 values for the MEK inhibitor, AZD6244, across the 152 cell lines. Below the histogram, the individual IC50 values are plotted using cyan squares (sensitive cell lines) and blue circles (resistant cell lines). The panel on the left depicts the histogram for the expression levels of gene ZBED2. To the right of the histogram, the individual expression levels are plotted using red plus signs (upregulated), yellow crosses (normal expression) and orange stars (downregulated). The scatter plot depicts the IC50 values and gene expression for each cell line. In this case, there are significantly many cell lines that show resistance to AZD6244 and are upregulated for ZBED2. These cell lines are found in the top-right area of the scatter plot and are indicated by red plus signs inside of blue circles. The MED12 knockdown signature contains a significantly large number of such genes indicating the potential predictive value of this signature.

FIG. 11 Breast cancer neo-adjuvant chemotherapy response rates of patients with a MED12 wt-like cancer and a MED12KD-like cancer. pCR, pathological complete response; RD, residual disease.

FIG. 12. MED12KD signature based on the 41 highest ranked genes (41-set, Table 1) predicts drug responses to cancer therapies.

A) Kaplan-Meier analysis of disease specific survival (DSS) for a cohort of 117 CRC cancers with MED12KD like gene signature. Patients with MED12KD-like cancers treated with chemotherapy did not show significant DSS, when compared with untreated patients (red line, chemotherapy; black line, no treatment; p=0.76).

B) Kaplan-Meier analysis of disease specific survival (DSS) for a cohort of 153 CRC cancers with MED12 wt like gene signature. Patients with MED12 wt like cancers treated with chemotherapy showed significant DSS when compared with untreated patients (red line, chemotherapy; black line, no treatment; p=0.00026).

### DETAILED DESCRIPTION

The invention provides a method of typing a sample from an individual suffering from cancer, the method comprising determining a level of expression for a set of at least 5 genes that are selected from Table 1 in a relevant sample from the individual, whereby the sample comprises expression products from a cancer cell of the patient, comparing said determined level of expression of the set of genes to the level of expression of the set of genes in a reference sample, and typing said sample based on the comparison of the determined levels of expression.

The levels of expression of the genes listed in Table 1 were found to be indicative of the activity of a component of the transcriptional MEDIATOR complex, MED12. It is noted that MED12 suppression often confers a slow-growth phenotype to cancer cells. However, near-complete suppression of MED12 is not tolerated by most cells. Thus, suppression of MED12 may not confer a selective advantage in the absence of drug, but may only become a benefit to the cancer cells when undergoing drug selection pressure. Consistent with this, it was observed that PC9 NSCLC, A375 melanoma and Huh-7 HCC cells are growth-inhibited by downmodulation of MED12, but this turns into a proliferative advantage when exposed to EGFR, BRAF or MEK inhibitors or the multikinase inhibitor sorafenib (FIG. 2G). It is shown that the changes of the levels of expression of the genes listed in Table 1 triggered by MED12 suppression are prognostic for disease outcome in colon cancer (FIG. 10) and in breast cancer (FIG. 11). In addition, changes of the levels of expression of the genes listed in Table 1 triggered by MED12 suppression are predictive for methotrexate-, camptothecin-, doxorubicin-, and vinblastin-resistance in cell lines.

MED12 is a component of the MEDIATOR transcriptional adapter complex that serves as a molecular bridge between the basal transcription machinery and its upstream activators (Conaway et al., 2005. TIBS 30: 250-255). More specifically, MED12 is a subunit of the “kinase” module of the MEDIATOR complex, which also contains MED13, CYCLIN C and CDK8, whose gene sequence is amplified in some 50% of colon cancers (Firestein et al, 2008. Nature 455: 547-551). The involvement of MEDIATOR components in responses to tyrosine kinase inhibitors (TKIs) was unexpected, as most of the known genes that influence responses to TKIs involve components of signaling pathways that act downstream or in parallel of these receptors. Applicants reconcile this apparent discrepancy by demonstrating that part of MED12 also resides in the cytosol, where it interacts with the TGFbeta type II receptor to inhibit its activity. Consequently, downregulation of MED12 by RNAi strongly activates TGFbeta signaling, as evidenced by phosphorylation of SMAD2 and induction of many canonical TGFbeta target genes. Activation of TGFbeta signaling has been linked previously to activation of ERK signaling (reviewed by (Zhang, 2009. Cell Research 19: 128-139)).

The inventors established that MED12 plays a role in regulating TGFbeta receptor signaling, which was found to be the major mechanism for induction of drug resistance by suppression of MED12. The results provided in the Examples show that cytoplasmic MED12 interacts with TGFbeta receptor type II (TGFBR2) and prevents the receptor from reaching the cell surface. TGFBR2 is made as an approximately 60 kDa primary polypeptide, which undergoes several sequential glycosylation steps in the Endoplasmic Reticulum and Golgi before reaching the cell surface. Consistent with this, 60 kDa, 70 kDa and 80-100 kDa forms of TGFBR2 have been identified. Endoglycosidase H (which cleaves asparagine-linked mannose rich oligosaccharides added in the ER, but not highly processed complex oligosaccharides formed in the Golgi complex) deglycosylates only the 70 kDa form of TGFBR2, while the enzyme PNGase F deglycosylates the mature form of TGFBR2, consistent with this being the Golgi-modified form of TGFBR2 (FIG. 6C). It was found that MED12 preferentially associates with the 60 kDa and 70 kDa forms of TGFBR2, but not with the mature (80-100 kDa) form of TGFBR2. This is consistent with the observation that 125-I-TGFbeta affinity labeled TGFBR2 localized at cell surface could not be co-immunoprecipitated with MED12 antibodies (data not shown). Together, these data demonstrate that MED12 interacts in the cytosol with unglycosylated and partially glycosylated TGFBR2, but not with the mature TGFBR2 at cell surface. This in turn indicates that cytoplasmic MED12 interferes with the proper glycosylation of TGFBR2 and hence blocks cell surface expression of the receptor.

Table 1 comprises a total of 252 genes, of which the first 234 genes are upregulated in cells with increased TGFbeta pathway activity by downmodulation of MED12, while 18 genes, including MED12, are downregulated in cells with increased TGFbeta pathway activity by downmodulation of MED12.

The term cancer, as used herein, refers to a benign tumor that, over time, may progress to become malignant, a malignant primary or metastasized tumor. Examples thereof include, but are not limited to, an adenoma, a carcinoma; a sarcoma, a lymphoma, a leukemia, or a myeloma.

The term “a relevant sample comprising expression products from a cancer cell of the patient” refers to a sample of the individual in which expression products of a cancer cell are present. Said sample is derived, for example, from a blood sample comprising cancer cells such as lymphoma cells, or derived from a primary or metastasized tumor, for example a breast cancer or colon cancer. A sample comprising expression products from a cancer cell of an individual suffering from cancer is provided after the removal of all or part of a cancerous growth from the individual, for example after biopsy. For example, a sample comprising expression products may be obtained from a needle biopsy sample or from a tissue sample comprising cancer cells that was previously removed by surgery. The surgical step of removing a relevant tissue sample, preferably a part of the cancer, from an individual is not part of a method according to the invention. It is preferred that at least 10% of the cells or tissue from which a relevant sample comprising expression products is derived, are cancer cells, more preferred at least 20%, more preferred at least 30%, more preferred at least 50%.

A further preferred set of genes according to the invention comprises at least six of the genes that are selected from Table 1, more preferred at least seven of the genes that are selected from Table 1, more preferred at least eight of the genes that are selected from Table 1, more preferred at least nine of the genes that are selected from Table 1, more preferred at least ten of the genes that are selected from Table 1, more preferred at least fifteen of the genes that are selected from Table 1, more preferred at least twenty of the genes that are selected from Table 1, more preferred at least twenty-five of the genes that are selected from Table 1, more preferred at least thirty of the genes that are selected from Table 1, more preferred at least forty of the genes that are selected from Table 1, more preferred at least forty-one of the genes that are selected from Table 1, more preferred at least forty-six genes of the genes that are selected from Table 1, more preferred at least fifty of the genes that are selected from Table 1, more preferred at least sixty of the genes that are selected from Table 1, more preferred at least seventy of the genes that are selected from Table 1, more preferred at least eighty of the genes that are selected from Table 1, more preferred hundred of the genes that are selected from Table 1, more preferred hundred-fifty of the genes that are selected from Table 1, more preferred two hundred thirty four genes; more preferred all two-hundred fifty two genes that are selected from Table 1, more preferred all of the genes that are selected from Table 1.

The genes that are rank-ordered 1-46 in Table 1 were rank-ordered according to the agreement of the outcome of typing of a sample with the individual genes to the outcome of the typing of a sample with the set of 46 genes. Similarly, the genes that are rank-ordered 47-234 and the genes that are rank-ordered 235-254 in Table 1 were rank-ordered according to the agreement of the outcome of typing of a sample with the individual genes to the outcome of the typing of a sample with the set of 254 genes.

A further preferred set of genes according to the invention comprises at least five genes of Table 1 that are rank-ordered 1-5. A further preferred set of genes according to the invention comprises at least ten genes of Table 1 that are rank-ordered 1-10, more preferred at least forty-one genes listed in Table 1 that are rank-ordered 1-41; more preferred at least forty-six genes listed in Table 1 that are rank-ordered 1-46; more preferred at least fifty genes listed in Table 1 that are rank-ordered 1-50; more preferred at least hundred genes listed in Table 1 that are rank-ordered 1-100; more preferred all 234 genes that are upregulated in cells with increased TGFbeta pathway activity by downmodulation of MED12, more preferred all 252 genes listed in Table 1.

A preferred set of genes comprises the forty-one genes listed in Table 1 having rank-order 1-41 with gene symbols LGALS1, EMP3, SPOCK1, TAGLN, CTGF, CDH2, TMEM45A, TNC, ECM1, DKK3, MAP1B, MYLK, SERPINE1, TIMP2, TUBB6, LAMB3, ANXA1, GBP2, PTHLH, CLIC3, GBP1, LAMA3, ABCA3, CTSE, CD55, SAMD9, HBEGF, MFI2, PPL, PTPN21, RND1, MICAL2, QPCT, TNNC1, RASAL2, GRAMD1B, CAMK2N1, THSD4, ARL14, ABAT and SEMA3C.

The methods and means of the instant invention further provide methods and means wherein a measurement of increased expression of a TGFbeta pathway gene and/or the measurement of an activating mutation in a TGFbeta pathway gene in one or more cancer cells of a cancer of a patient identifies the cancer as one that has a high risk of being or becoming resistant to anti-cancer treatment.

A measurement of increased expression of a TGFbeta pathway gene, and/or the measurement of a modulating mutation in a TGFbeta pathway gene in one or more cancer cells of a patient indicates the patient may be or become resistant to anti-cancer treatment. Said patient may benefit from treatment with an inhibitor of the TGFbeta pathway (e.g., a TGFbeta inhibitor and/or inhibitor of one or more downstream signaling proteins in the TGFbeta pathway), either alone or in combination with one or more chemotherapeutic agents selected from the list of chemotherapeutic compounds provided herein below.

Increased expression of a TGFbeta pathway genes can be determined by any of the methods known in the art, including DNA microarrays, qPCR and next generation sequencing, as is described herein below for determining the level of expression of a set of genes listed in Table 1. A modulating mutation in a TGFbeta pathway gene that results in increased TGFbeta pathway activity is, for example an activating mutation in an activator of the pathway, for example SMAD2 or SMAD4, or an inactivating mutation in a repressor of the pathway, for example c-Ski or c-SnON. Said mutation may be determined can be determined by analysis of the encoded protein by, for example, protein sequence determination, two dimensional gel electrophoresis, multidimensional protein identification technology, ELISA, liquid chromatography-mass spectrometry (LC-MS), matrix-assisted laser desorption/ionization time-of-flight mass spectrometry (MALDI-TOF), or the use of antibodies that interact with either a non-mutated normal form, or with a mutated variant form of PIK3CA. Alternatively, a nucleotide sequence of a TGFbeta pathway gene is determined by any method known in the art, including but not limited to sequence analysis of a genomic region encoding PIK3CA and sequence analysis of a mRNA product or a derivative of a mRNA product such as a cDNA product, by any method known in the art, including but not limited to dideoxy sequencing, matrix-assisted laser desorption/ionization time-of-flight mass spectrometry, and sequencing by hybridization, including hybridization with sequence-specific oligonucleotides and hybridization to oligonucleotide arrays. In addition, mutation analysis methods such as single stranded conformation polymorphism, DNA heteroduplex analysis, denaturing gradient gel electrophoresis and thermal gradient gel electrophoresis may be used to determine the presence or absence of a modulating mutation in a TGFbeta pathway gene.

Increased TGFbeta pathway activity by, for example, increased expression of a TGFbeta pathway gene and/or the presence of a modulating mutation in a TGFbeta pathway gene can further be determined by determining the levels of expression of at least five of the genes listed in Table 1 in a relevant sample comprising cancer cells of an individual, and comparing the determined level of expression with the level of expression of the at least five of the genes listed in Table 1 in a reference sample.

The terms “TGFbeta pathway” and “TGFbeta pathway gene” refer to any gene encoding for a protein in the TGFbeta signaling pathway, including Type I and Type II receptor; SMAD (Sma and Mad Related Family) family of signal transducers, SMAD2, SMAD3, SMAD4, SMAD6; SMAD Anchor for Receptor Activation (SARA); c-Ski and c-SnON; phosphatidylinositol-3-kinase (PI3K); protein phosphatase-2A (PP2A); transcriptional coactivators and corepressors like p300 and CREB Binding Protein (CBP); Forkhead Activin Signal Transducer-2 (FAST2); RhoA; Ras; TGFbeta Activated Kinase (TAK); TAK1 Binding Protein (TAB1); Xenopus Inhibitor of Apoptosis (XIAP); Haematopoietic Progenitor Kinase-1 (HPK1); MAP kinase Kinase and MAPK/ERK Kinase pathways, including JNK/SPAK, p38, and ERK1/2.

Because of its critical role in cell fate determination, TGFbeta signalling is subject to many levels of positive and negative regulation, targeting both the receptors and the intracellular mediators. Among the negative regulators of SMAD function are two highly conserved members of the Ski family of proto-oncoproteins c-Ski and c-SnON that antagonizes TGFbeta signalling through direct interactions with the SMAD2/SMAD3 and SMAD4 and later degrade releasing SMADs to regulate transcription.

A sample from an individual suffering from cancer comprising expression products from a cancer of the patient can be obtained in numerous ways, as is known to a skilled person. For example, the sample can be freshly prepared from cells or a tissue sample at the moment of harvesting, or it can be prepared from samples that are stored at −70° C. until processed for sample preparation. Alternatively, tissues or biopsies can be stored under conditions that preserve the quality of the protein or RNA. Examples of these preservative conditions include fixation using e.g. formaline and paraffin embedding, the addition of RNase inhibitors such as RNAsin® (Pharmingen) or RNasecure® (Ambion), aquous solutions such as RNAlater® (Assuragen; U.S. Ser. No. 06/204,375), Hepes-Glutamic acid buffer mediated Organic solvent Protection Effect (HOPE; DE10021390), and RCL2 (Alphelys; WO04083369), and non-aquous solutions such as Universal Molecular Fixative (Sakura Finetek USA Inc.; U.S. Pat. No. 7,138,226).

Said expression products are protein expression products or, preferably, RNA expression products. A sample from an individual suffering from cancer comprising protein expression products from a cancer of the patient can be obtained in numerous ways, as is known to a skilled person. For example, proteins can be isolated from a sample using, for example, cell disruption and extraction of cellular contents. Suitable methods and means are known in the art, such as dounce pestles and sonication methods. In addition, preferred methods include reagent-based lysis methods using detergents. These methods not only lyse cells but also solubilize proteins. Cell disruption may be followed by methods for enrichment of specific proteins, including subcellular fractionation and depletion of high abundant proteins. Differences in protein expression between a sample from an individual suffering from cancer and a reference sample is studied, for example, by two-dimensional (2D) gel electrophoresis and/or mass spectrometry techniques such as, for example, electrospray ionization and matrix-assisted laser desorption ionization.

RNA may be isolated from a sample by any technique known in the art, including but not limited to Trizol (Invitrogen; Carlsbad, Calif.), RNAqueous® (Applied Biosystems/Ambion, Austin, Tx), Qiazol® (Qiagen, Hilden, Germany), RNeasy Isolation Kit (Qiagen, Hilden, Germany) Agilent Total RNA Isolation Kits (Agilent; Santa Clara, Calif.), RNA-Bee® (Tel-Test. Friendswood, Tex.), and Maxwell™ Total RNA Purification Kit (Promega; Madison, Wis.). A preferred RNA isolation procedure involves the use of Qiazol® (Qiagen, Hilden, Germany). A further preferred RNA isolation procedure involves the use of the Qiagen RNeasy FFPE RNA isolation Kits (Qiagen, Hilden, Germany). RNA can be extracted from a whole sample or from a portion of a sample generated from the cell sample by, for example, section or laser dissection.

The level of RNA expression of a signature gene according to the invention can be determined by any method known in the art. Methods to determine RNA levels of genes are known to a skilled person and include, but are not limited to, Northern blotting, quantitative Polymerase chain reaction (qPCR), microarray analysis and RNA sequencing. The term qPCR refers to a method that allows amplification of relatively short (usually 50 to 500 base pairs) of DNA sequences. The term “qPCR” is often used as an equivalent to the term “real-time PCR”, which allows quantification of starting amounts of DNA, cDNA, or RNA templates by detection of a fluorescent reporter molecule that increases as PCR product accumulates with each cycle of amplification. In order to quantitatively measure messenger RNA (mRNA), the method is extended using reverse transcriptase to convert mRNA into complementary DNA (cDNA) which is then amplified by PCR. The amount of product that is amplified can be quantified using, for example, TaqMan® (Applied Biosystems, Foster City, Calif., USA), Molecular Beacons, Scorpions® and SYBR® Green (Molecular Probes). Quantitative Nucleic acid sequence based amplification (qNASBA) can be used as an alternative for qPCR.

Different amplification methods, known to a skilled artisan, can be employed for qPCR, including but not limited to PCR, rolling circle amplification, nucleic acid sequence-based amplification, transcription mediated amplification, and linear RNA amplification. In addition, for the simultaneous detection of specific multiple nucleic acid gene expression products, qPCR methods such as reverse transcriptase-multiplex ligation-dependent amplification (rtMLPA), which accurately quantifies up to 45 transcripts of interest in a one-tube assay (Eldering et al., Nucleic Acids Res 2003; 31: e153) are preferably employed.

A further preferred method for determining a level of RNA expression comprises next-generation sequencing, involving isolation and fragmentation of RNA followed by library creation and sequencing of the resulting cDNAs. Said RNA is preferably enriched for messenger RNA (mRNA) and/or depleted of rRNA. An index is preferably ligated prior to an amplification step, allowing multiplex amplification of several samples prior to the sequencing. Next generation sequencing platforms are available from, for example, Pacific Biosciences, Oxford Nanopore Technologies, Complete Genomics, Illumina and Applied Biosystems.

A further preferred method for determining a level of RNA expression comprises microarray analysis. Microarray-based analyses involve the use of selected biomolecules that are immobilized on a surface. A microarray usually comprises nucleic acid molecules, termed probes, which are able to hybridize to nucleic acid expression products or their complementary sequences. The probes are exposed to labeled sample nucleic acid and hybridized, where after the abundance of nucleic acid expression products in the sample that are complementary to a probe is determined. The probes on a microarray may comprise DNA sequences, RNA sequences, or copolymer sequences of DNA and RNA. The probes may also comprise DNA and/or RNA analogues such as, for example, nucleotide analogues or peptide nucleic acid molecules (PNA), or combinations thereof. The sequences of the probes may be full or partial fragments of genomic DNA. The sequences may also be in vitro synthesized nucleotide sequences, such as synthetic oligonucleotide sequences.

For microarray analysis, a hybridization mixture is prepared by extracting and labelling of RNA expression products. The extracted RNA is preferably converted into a labelled sample comprising either complementary DNA (cDNA) or cRNA using a reverse-transcriptase enzyme and labelled nucleotides. A preferred labelling introduces fluorescently-labelled nucleotides such as, but not limited to, cyanine-3-CTP or cyanine-5-CTP. Examples of labelling methods that are known in the art include Low RNA Input Fluorescent Labelling Kit (Agilent Technologies), Agilent's Genomic DNA labelling Kits (Agilent Technologies), MessageAmp Kit (Ambion) and Microarray Labelling Kit (Stratagene).

A probe preferably specifically hybridizes to an expression product of a gene. A probe is specific when it comprises a continuous stretch of nucleotides that are complementary to a nucleotide sequence of a RNA product of said gene, or a cDNA product thereof. The term complementary is known in the art and refers to a sequence that is related by base-pairing rules to the sequence that is to be detected. It is preferred that the sequence of the probe is carefully designed to minimize nonspecific hybridization to said probe. It is further preferred that the probe is or mimics a single stranded nucleic acid molecule. The length of said complementary continuous stretch of nucleotides can vary between 15 bases and several kilo bases, and is preferably between 20 bases and 1 kilobase, more preferred between 40 and 100 bases, and most preferred 60 nucleotides. A most preferred probe comprises a continuous stretch of 60 nucleotides that are identical to a stretch of nucleotides of a RNA product of a gene, or a cDNA product thereof.

The probe preferably specifically hybridizes to an expression product of a gene under stringent hybridization conditions. The term “stringent hybridization conditions” refers to conditions under which a probe will hybridize to its target subsequence, typically in a complex mixture of nucleic acids, but to essentially no other sequences. Stringent conditions are sequence-dependent and will be different in different circumstances. Longer sequences hybridize specifically at higher temperatures. An extensive guide to the hybridization of nucleic acids is found in Thijssen (Thijssen, 1993. In: Laboratory Techniques in Biochemistry and Molecular Biology. Elsevier). Generally, stringent conditions are selected to be about 5-10° C. lower than the thermal melting point (Tm) for the specific sequence at a defined ionic strength pH. The Tm is the temperature (under defined ionic strength, pH, and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions are often: 50% formamide, 5×SSC, and 1% SDS, incubating at 42° C., or, 5×SSC, 1% SDS, incubating at 65° C., with wash in 0.2×SSC, and 0.1% SDS at 65° C. Additional guidelines for determining hybridization parameters are provided in numerous references, e.g. Current Protocols in Molecular Biology, eds. Ausubel, et al. 1995. 4th edition, John Wiley and Sons Inc., New York, N.Y.

The level of expression of a set of genes that are selected from Table 1, preferably RNA expression products, in a relevant sample is preferably normalized to correct for systemic bias. Systemic bias in microarray-mediated analyses results in variation by, for example, differences in overall performance, which can be due to inconsistencies in array fabrication, staining and scanning, and variation between labeled cRNA or cDNA samples due to variations in purity. Systemic bias can be introduced during the handling of the sample in a microarray experiment. To reduce systemic bias, the determined RNA levels are preferably corrected for background non-specific hybridization and normalized using, for example, Feature Extraction software (Agilent Technologies). Other methods or software that are or will be known to a person of ordinary skill in the art, such as a dye swap experiment (Martin-Magniette et al., Bioinformatics 21:1995-2000 (2005)) can also be applied to normalize differences introduced by dye bias.

Conventional methods for normalization of array data include global analysis, which is based on the assumption that the majority of genetic markers on an array are not differentially expressed between samples [Yang et al., Nucl Acids Res 30: 15 (2002)]. As an alternative, an external “spike” RNA or DNA can be added to all samples, whereby probes that are complementary to the spiked RNA are present on the array.

Alternatively, the array may comprise specific probes that are used for normalization. These probes preferably detect RNA products from housekeeping genes such as glyceraldehyde-3-phosphate dehydrogenase and 18S rRNA levels, of which the RNA level is thought to be constant in a given cell and independent from the developmental stage or prognosis of said cell. As an alternative, a set of genes has been described of which the level of expression was found to be constant between different samples (WO2008/039071; which is hereby included by reference). Their constant level of expression allows their use for normalization of gene expression hybridization data, especially when arrays with a limited number of probes (comprising between 2 and 12.000 probes) are used.

Cancers for which the prognostic methods and compositions of the instant invention may provide predictive results for resistance to anti-cancer treatment include cancers such as breast cancer (e.g., BRCA-1 deficient, stage-III HER2-negative, luminal type, basal type, ERBB2 type, ER/PR positive, HER2 positive, ductal carcinoma, lobular carcinoma), ovarian cancer (e.g., BRCA-1 deficient, epithelial ovarian cancer), lung cancer (e.g., non-small-cell lung cancer or small cell lung cancer, metastatic non-small cell lung cancer), liver cancer (e.g., hepatocellular carcinoma), head and neck cancer (e.g., metastatic squamous cell carcinoma of the head and neck (HNSCC), squamous cell carcinoma, laryngeal cancer, hypopharyngeal cancer, oropharyngeal cancer, and oral cavity cancer), bladder cancer (e.g., transitional cell carcinoma of the bladder), and colorectal cancer (e.g., advanced (non-resectable locally advanced or metastatic) colorectal cancer). Other cancers for which the methods and compositions of the invention may provide predictive treatment include cervical cancer (e.g., recurrent and stage IVB), mesothelioma, solid cancers (e.g., advanced solid cancers), renal cell carcinoma (e.g., advanced renal cell carcinoma), stomach cancer, sarcoma, prostate cancer (e.g., hormone refractory prostate cancer), melanoma, thyroid cancer (e.g., papillary thyroid cancer), brain cancer, adenocarcinoma, subependymal giant cell astrocytoma, endometrial cancer, glioma, glioblastoma, and other cancers that have metastasized to the brain, esophageal cancer, neuroblastoma, hematological cancers, and lymphoma.

Said cancer is typically selected from colorectal cancer, lung cancer, liver cancer, prostate cancer and breast cancer.

The term “typing of a sample”, as used in this application, refers to determining characteristics of a sample. Said characteristics include the determination of variables such as the level of expression of a set of genes in the sample. The typing of a sample may assist in the classification of that sample. Typing according to the instant invention preferably assists in the prediction of whether a cancer is resistant to anti-cancer therapy.

Typing of a sample can be performed in various ways. In a preferred method, said typing indicates that the cancer has a high risk of being or becoming resistant to anti-cancer treatment when the level of expression of the set of genes in a relevant sample from the individual is altered, when compared to the level of expression of the set of genes in a relevant reference sample or reference population. A reference sample preferably is a sample comprising expression products from a related or an unrelated source. A preferred reference sample comprises expression products from a collection of cell lines, representing different tissues, or from a collection of tissue samples with and without a cancer, or from samples comprising resistant and non-resistant cancers, or from samples comprising different stages of a cancer.

In a preferred method according to the invention, said typing is used to indicate that the individual has a high risk of being or becoming resistant to anti-cancer treatment ((MED12-knock down like; MED12kd-like) when the level of expression of the set of genes in a relevant sample from the individual is induced (gene rank numbers 1-234 of Table 1), or as having a low risk of being or becoming resistant to anti-cancer treatment (MED12wild type; MED12 wt) when the level of expression of the set of genes in a relevant sample from the individual is not induced (gene rank numbers 1-234 of Table 1), whereby the level of expression of the set of genes is compared to the level of expression of the set of genes in a reference sample or reference population.

In a further preferred method according to the invention, said typing is used to indicate that the individual has a low risk of being or becoming resistant to anti-cancer treatment when the level of expression of the set of genes in a relevant sample from the individual is induced (gene rank numbers 235-252) of Table 1), or as having a high risk of being or becoming resistant to anti-cancer treatment when the level of expression of the set of genes in a relevant sample from the individual is not induced (gene rank numbers 235-252 of Table 1), whereby the level of expression of the set of genes is compared to the level of expression of the set of genes in a reference sample or reference population.

In yet a further preferred method, said typing is used to indicate that the individual has a low risk of being or becoming resistant to anti-cancer treatment when the level of expression of the set of genes in a relevant sample from the individual is not induced (gene rank numbers 1-234 of Table 1) and/or when the level of expression of the set of genes in a relevant sample from the individual is induced (gene rank numbers 235-252) of Table 1), or as having a high risk of being or becoming resistant to anti-cancer treatment when the level of expression of the set of genes in a relevant sample from the individual is induced (gene rank numbers 1-234 of Table 1) and/or when the level of expression of the set of genes in a relevant sample from the individual is not induced (gene rank numbers 235-252 of Table 1), whereby the level of expression of the set of genes is compared to the level of expression of the set of genes in a reference sample or reference population.

In a preferred method, the level of expression of a set of genes, selected from the genes that are rank ordered 1-234, is combined, for example by summation or averaging (e.g. by calculation of the sum, average, median, modus), or by counting the number/percentage of induced genes, and used for typing of a sample. The actual threshold for typing a sample as MED12kd-like or MED12 wt may depend on the individual cancer sample. For example, a sample comprising expression products from a colon cancer cell could be typed as MED12-like if the level of expression of more than 50% of the set of genes, selected from the genes that are rank ordered 1-234, was found to be induced, compared to a reference sample. A sample comprising expression products from a breast cancer cell could be typed as MED12-like if the level of expression of more than 75% of the set of genes, selected from the genes that are rank ordered 1-234, was found to be induced, compared to a reference sample.

In a further preferred method, the level of expression of a set of genes, selected from the genes that are rank ordered 235-252 or 1-252, is combined, for example by summation or averaging (e.g. by calculation of the sum, average, median, modus), or by counting the number/percentage of not-induced genes and used for typing of a sample.

In a further preferred method, the level of expression of a set of genes, selected from the genes that are rank ordered 1-46, is combined, for example by summation or averaging (e.g. by calculation of the sum, average, median, modus), or by counting the number/percentage of induced genes, and used for typing of a sample.

In a preferred method, a coefficient is determined, which is a measure of a similarity or dissimilarity of the level of expression of the set of genes in the sample with the reference sample. A number of different coefficients can be used for determining a correlation between the RNA expression level in an RNA sample from an individual and a reference sample. Preferred methods are parametric methods which assume a normal distribution of the data. One of these methods is the Pearson product-moment correlation coefficient, which is obtained by dividing the covariance of the two variables by the product of their standard deviations. Preferred methods comprise cosine-angle, un-centered correlation and, more preferred, cosine correlation (Fan et al., Conf Proc IEEE Eng Med Biol Soc. 5:4810-3 (2005)).

Preferably, said correlation with a reference sample or reference population is used to produce an overall similarity score for the set of genes that are used. A similarity score is a measure of the average correlation of expression levels of a set of genes, preferably RNA expression levels, between a sample from an individual and a reference sample or reference population. Said similarity score can, for example, be a numerical value between +1, indicative of a high correlation between the RNA expression level of the set of genes in a RNA sample of said individual and said reference sample, and −1, which is indicative of an inverse correlation and therefore indicative of having an increased risk of cancer recurrence (van't Veer et al., Nature 415: 484-5 (2002)).

A preferred method of typing an individual suffering from cancer comprises classifying said individual as having a high risk of being or becoming resistant to anti-cancer treatment or as having a low risk of being or becoming resistant to anti-cancer treatment. In a preferred method, a similarity value is determined between the expression levels of a set of genes listed in Table 1 in a sample from said individual and a level of expression from the same set of genes in a reference sample or reference population, and classifying said individual as having a high risk of being or becoming resistant to anti-cancer treatment if said similarity value is below a first similarity threshold value, and classifying said individual as having a low risk of being or becoming resistant to anti-cancer treatment if said similarity value exceeds said first similarity threshold value.

In a further preferred method, a similarity value is determined between the expression levels of a set of genes listed in Table 1 in a sample from said individual and a level of expression from the same set of genes in a reference sample or reference population, and classifying said individual as having a having a low risk of being or becoming resistant to anti-cancer treatment if said similarity value is below a first similarity threshold value, and classifying said individual as having a high risk of being or becoming resistant to anti-cancer treatment if said similarity value exceeds said first similarity threshold value.

The skilled person understands that said reference sample may refer to the average level of expression of the set of genes listed in Table 1 in one or more cancers or cell lines known to be sensitive to the anti-cancer treatment, or to the average level of expression of the set of genes listed in Table 1 in one or more cancers or cell lines known to be resistant to the anti-cancer treatment or known to be sensitive to the anti-cancer treatment. As an alternative, the reference sample may refer to the average level of expression of the set of genes listed in Table 1 in a mixture of cancers or a mixture of cell lines that are known to be sensitive and/or resistant to the anti-cancer treatment.

The terms “resistant,” or “resistant to anti-cancer treatment” in the context of treatment of a cancer cell with a chemotherapeutic agent mean that the chemotherapeutic agent is not likely to have an optimal effect on the cancer cell, meaning that the effect of the chemotherapeutic agent on one or more cancer cells is reduced or absent. The terms, therefore, also cover a tumor or cancer that is less sensitive to a chemotherapeutic agent, but not completely resistant to it.

A cancer cell in a patient that demonstrates resistance to an anti-cancer treatment with a chemotherapeutic agent is either a cell that has never been treated with the anti-cancer treatment and which demonstrates resistance to the anti-cancer drug or drugs once treatment has begun, termed primary resistance, or a cancer cell in a patient that has been treated with the anti-cancer treatment and acquires resistance during of after treatment, termed “secondary resistance”.

It was found by the present inventors that downmodulation of MED12 in a cancer cell, as indicated by the level of expression of a set of genes indicated in Table 1, is indicative of a cancer cell that is resistant to anti-cancer treatment. Said resistance is either primary of secondary.

Anti-cancer treatment in general is directed to disturbing cell multiplication or normal functioning, DNA synthesis or chromosomal migration, and to blocking or changing RNA and protein metabolism. Anti-cancer treatment includes the use of a chemotherapeutic agent such as an alkylating agent such as nitrogen mustard, e.g. cyclophosphamide, mechlorethamine or mustine, uramustine or uracil mustard, melphalan, chlorambucil, ifosfamide; a nitrosourea such as carmustine, lomustine, streptozocin; an alkyl sulfonate such as busulfan, an ethylenime such as thiotepa and analogues thereof, a hydrazine/triazine such as dacarbazine, altretamine, mitozolomide, temozolomide, altretamine, procarbazine, dacarbazine and temozolomide, which are capable of causing DNA damage; an intercalating agent such as a platinum agent like cisplatin, carboplatin, nedaplatin, oxaliplatin and satraplatin; an antibiotic such as an anthracycline such as doxorubicin, daunorubicin, epirubicin and idarubicin; mitomycin-C, dactinomycin, bleomycin, adriamycin, mithramycin; an antimetabolite such as capecitabine and 5-fluorouracil, gemcitabine, a folate analogue such as methotrexate, hydroxyurea, mercaptopurine, thioguanine; a mitostatic agent such as eribulin, ixabepilone, irinotecan, vincristine, mitoxantrone, vinorelbine and a taxane such as paclitaxel and docetaxel; a receptor tyrosine kinase inhibitor such as gefitinib, erlotinib, EKB-569, lapatinib, CI-1033, cetuximab, panitumumab, PKI-166, AEE788, sunitinib, sorafenib, dasatinib, nilotinib, pazopanib, vandetaniv, cediranib, afatinib, motesanib, CUDC-101, and imatinib mesylate; a MEK inhibitor including CKI-27, RO-4987655, RO-5126766, PD-0325901, WX-554, AZD-8330, G-573, RG-7167, SF-2626, GDC-0623, RO-5068760, and AD-GL0001; a B-RAF inhibitor including CEP-32496, vemurafenib, GSK-2118436, ARQ-736, RG-7256, XL-281, DCC-2036, GDC-0879, AZ628, and an antibody fragment EphB4/Raf inhibitor; a serine/threonine kinase receptor inhibitor, including an Alk-1 inhibitor such as crizotinib, ASP-3026, LDK378, AF802, and CEP37440, and combinations thereof.

Said anti-cancer treatment is preferably selected from a platinum agent like cisplatin, carboplatin, oxaliplatin and satraplatin; taxane including paclitaxel and docetaxel, doxorubicin, daunorubicin, epirubicin, cyclophosphamide, 5-fluorouracil, gemcitabine, eribulin, ixabepilone, methotrexate, mitomycin-C, mitoxantrone, vinorelbine, thiotepa, vincristine, capecitabine, a receptor tyrosine kinase inhibitor and/or irinotecan, and combinations thereof.

Said anti-cancer treatment is preferably selected from a platinum agent like cisplatin, carboplatin, oxaliplatin and satraplatin; taxane including paclitaxel and docetaxel, doxorubicin, daunorubicin, epirubicin, cyclophosphamide, 5-fluorouracil, gemcitabine, eribulin, ixabepilone, methotrexate, mitomycin-C, mitoxantrone, vinorelbine, thiotepa, vincristine, capecitabine, and/or irinotecan, and combinations thereof.

It is further preferred that a method of typing a sample from an individual suffering from cancer further comprises determining a strategy for treatment of the patient. Said sample is obtained from a patient either prior to the anti-cancer treatment, or during or after said anti-cancer treatment. Typing of a sample from an individual suffering from cancer according to the methods of the invention, and classifying said cancer as having a high risk of being or becoming resistant to anti-cancer treatment identifies the individual as one that may benefit from treatment with an inhibitor of the TGFbeta pathway, either alone or, preferably, in combination with said anti-cancer treatment.

The methods and means of the instant invention further provide methods and means wherein a measurement of increased expression of a TGFbeta pathway gene and/or the measurement of an activating mutation in a TGFbeta pathway gene in one or more cancer cells of a cancer of a patient identifies the cancer as one that may benefit from treatment with an inhibitor of the TGFbeta pathway (e.g., a TGFbeta inhibitor and/or inhibitor of one or more downstream signaling proteins in the TGFbeta pathway), either alone or in combination with one or more chemotherapeutic agents selected from the list of chemotherapeutic compounds provided herein below.

A measurement of increased expression of a TGFbeta pathway gene, and/or the measurement of an activating mutation in a TGFbeta pathway gene in one or more cancer cells of a patient indicates the patient may be resistant to anti-cancer treatment. Said patient may benefit from treatment with an inhibitor of the TGFbeta pathway (e.g., a TGFbeta inhibitor and/or inhibitor of one or more downstream signaling proteins in the TGFbeta pathway), either alone or in combination with one or more chemotherapeutic agents selected from the list of chemotherapeutic compounds that are provided herein.

An inhibitor of the TGFbeta pathway comprises neutralizing antibodies to TGFbeta or TGFbeta receptors or by the therapeutic use of proteins or synthetic compounds, which bind TGFbeta, and thereby prevent its binding to TGFbeta receptors. Antibodies that have been described and are considered as TGFbeta blockers include Metelimumat, a monoclonal antibody to TGFbeta 1, which inhibits the function of TGFbeta (from Cambridge Antibody Technology); GC-1008, a humanized monoclonal antibody to TGFbeta 1, TGFbeta 2 and TGFbeta 3 (Genzyme, Inc), lerdelimumat, a monoclonal antibody to TGFbeta 1 (from Cambridge Antibody Technology), and 1D11 and 2G7 (Genentech), which are high-affinity monoclonal antibodies capable of neutralizing all three major mammalian isoforms of TGFbeta (i.e., 1, 2, and 3). In addition, peptides comprising parts of TGFbeta can be used as inhibitor of the TGFbeta pathway according to the invention including, for example peptides comprising amino acids 41-65 of TGFbeta 1.

TGFbeta binding can also be inhibited with soluble TGFbeta receptors. An example of such inhibitors of the TGFbeta pathway is the soluble TGFbeta type III receptor fusion proteins comprising all or a portion of the Fc tail of human IgG covalently linked to all or an active portion of a splice variant of the extracellular domain of human TGFbeta or activin type Il or type Il B-receptor (see WO 2005/028517, Gen Hospital Corp.). Interference with TGFbeta mediated signaling is further achieved by (1) the overexpression of SMAD7 by virus-mediated gene transfer, which results in the inhibition of the TGFbeta receptor mediated phosphorylation of SMAD transcription factors required for the transcription of TGFbeta regulated genes, and (2) small organic compounds, which block TGFbeta receptor kinases, and thereby block the activation of SMAD proteins. Such small organic compounds, which are considered as inhibitor of the TGFbeta pathway according to the invention, comprise novel pyrazole compounds and related dihydropyrrolo pyrazoles, and are highly potent inhibitors of the TGFbeta receptor kinase by binding to the ATP-binding pocket of the kinase. Such compounds with a high degree of selectivity for the TGFbeta receptor, and their in vivo application are described by Peng et al., 2005 (Peng et al., 2005. Biochemistry 44 2293-2304). Another example of a inhibitor of the TGFbeta pathway according to the invention is LY2157299 (4-(2-(6-methylpyridin-2-yl)-5,6-dihydro-4H-pyrrolo[1,2-b]pyrazol-3-yl)quinoline-6-carboxamide) from Eli Lilly & Company, which modulates TGFbeta R1 function by inhibiting SMAD2 phosphorylation. Still further compounds that are considered as inhibitor of the TGFbeta pathway according to the invention include
(1) quinazoline derivatives (WO 2004/081009, Millennium Pharmaceuticals Inc);
(2) 4-(pyridin-4-ylamino)-quinoline derivatives (WO2004/112710, Millennium Pharmaceuticals Inc.),
(3) Thiazole derivatives (WO 02/62793, Glaxo Group Ltd.);
(4) N-acetyl-seryl-aspartyl-lysyl-proline (Ac-SDKP) which inhibits effects of TGFbeta by interfering with nuclear translocation of SMAD proteins (Kanasaki et al., 2003. J. Am. Soc. Nephrol 14: 863-872).
(5) A cytoxazon derivate which inhibits the TGFbeta signal transduction pathway (WO2005/039570, RIKEN KK);
(6) Nitrogen-containing heterocyclic compounds and other aromatic organic compounds (WO2004/056352, Scios Inc.);
(7) Substituted p-benzoyloxy- or p-phenyloxycarbonyl-phenylamidines and -guanines, inhibitors for release, activation and synthesis of TGFbeta (JP 08333249, ONO Pharma Co.).
(8) Small interfering ribonucleic acid (siRNA) molecules which reduce expression of the TGFbeta type Il receptor (WO2005/019422, Univ. of Illinois Found.);
(9) TGFbeta 1 and TGFbeta 2 antisense constructs (from Antisense Pharma GmbH); and RNA ligand TGFbeta 1 selected from a group of 137 sequences (WO 1999/489004, Nexstar Pharma Inc.).

A preferred inhibitor of the TGFbeta pathway is or comprises LY2157299. LY2157299 is a small molecule inhibitor targeting both TGFbeta R1 and TGFbeta R2, and is currently being evaluated in clinical trials for the treatment of several cancer types. As is shown in the experimental data, control H3122 cells were compared to H3122 cells in which MED12 was downmodulated by RNAi (MED12KD). Crizotinib alone potently inhibited the proliferation of the control, but not of the MED12KD cells. LY2157299 monotherapy had little effect on all cells. However, strong synergy was seen when crizotinib was combined with LY2157299, consistent with the notion derived from the RNAi experiment that TGFbeta R2 inhibition restored the sensitivity of MED12KD cells to crizotinib. Importantly, the same synergistic response was also obtained when LY2157299 was combined with gefitinib to suppress proliferation of MED12KD PC9 cells (FIG. 9). Thus, the combination of a TGFbeta receptor inhibitor such as LY2157299 and anti-cancer treatment such as crizotini or gefitinib is a strategy for treating cancers with elevated TGFbeta signalling, such as cancers with reduced cytoplasmic MED12 activity.

The invention further provides a method for assigning treatment to an individual suffering from cancer, comprising (a) typing a relevant sample from the patient according to the method of the invention, (b) classifying said sample as having a high risk of being or becoming resistant to anti-cancer treatment or as having a low risk of being or becoming resistant to anti-cancer treatment, and (c) assigning anti-TGFbeta treatment to an individual of which the sample is classified as having a high risk of being or becoming resistant to anti-cancer treatment.

It is preferred that said anti-TGFbeta treatment is combined with said anti-cancer treatment. A preferred anti-TGFbeta treatment is or comprises administration of LY2157299 in a pharmaceutically acceptable formulation. Said anti-cancer treatment is preferably selected from one or more of a chemotherapeutic agent or other compound such as an alkylating agent such as nitrogen mustard, e.g. cyclophosphamide, mechlorethamine or mustine, uramustine or uracil mustard, melphalan, chlorambucil, ifosfamide; a nitrosourea such as carmustine, lomustine, streptozocin; an alkyl sulfonate such as busulfan, an ethylenime such as thiotepa and analogues thereof, a hydrazine/triazine such as dacarbazine, altretamine, mitozolomide, temozolomide, altretamine, procarbazine, dacarbazine and temozolomide, which are capable of causing DNA damage; an intercalating agent such as a platinum agent like cisplatin, carboplatin, nedaplatin, oxaliplatin and satraplatin; an antibiotic such as an anthracycline such as doxorubicin, daunorubicin, epirubicin and idarubicin; mitomycin-C, dactinomycin, bleomycin, adriamycin, mithramycin; an antimetabolite such as capecitabine and 5-fluorouracil, gemcitabine, a folate analogue such as methotrexate, hydroxyurea, mercaptopurine, thioguanine; a mitostatic agent such as eribulin, ixabepilone, irinotecan, vincristine, mitoxantrone, vinorelbine and a taxane such as paclitaxel and docetaxel; a receptor tyrosine kinase inhibitor such as gefitinib, erlotinib, EKB-569, lapatinib, CI-1033, cetuximab, panitumumab, PKI-166, AEE788, sunitinib, sorafenib, dasatinib, nilotinib, pazopanib, vandetaniv, cediranib, afatinib, motesanib, CUDC-101, and imatinib mesylate; a MEK inhibitor including CKI-27, RO-4987655, RO-5126766, PD-0325901, WX-554, AZD-8330, G-573, RG-7167, SF-2626, GDC-0623, RO-5068760, and AD-GL0001; a B-RAF inhibitor including CEP-32496, vemurafenib, GSK-2118436, ARQ-736, RG-7256, XL-281, DCC-2036, GDC-0879, AZ628, and an antibody fragment EphB4/Raf inhibitor; a serine/threonine kinase receptor inhibitor, including an Alk-1 inhibitor such as crizotinib, ASP-3026, LDK378, AF802, and CEP37440, and combinations thereof.

Said anti-cancer treatment is preferably selected from a platinum agent like cisplatin, carboplatin, oxaliplatin and satraplatin; taxane including paclitaxel and docetaxel, doxorubicin, daunorubicin, epirubicin, cyclophosphamide, 5-fluorouracil, gemcitabine, eribulin, ixabepilone, methotrexate, mitomycin-C, mitoxantrone, vinorelbine, thiotepa, vincristine, capecitabine, a receptor tyrosine kinase inhibitor and/or irinotecan.

Said anti-cancer treatment is preferably selected from a platinum agent like cisplatin, carboplatin, oxaliplatin and satraplatin; taxane including paclitaxel and docetaxel, doxorubicin, daunorubicin, epirubicin, cyclophosphamide, 5-fluorouracil, gemcitabine, eribulin, ixabepilone, methotrexate, mitomycin-C, mitoxantrone, vinorelbine, thiotepa, vincristine, capecitabine, and/or irinotecan.

### EXAMPLES

### Example 1

### General Materials and Methods

Crizotinib (S1068), NVP-TAE648 (S1108), gefitinib (S1025), erlotinib (S1023), PLX4032 (S1267), AZD6244 (S1008), PD0325901 (S1036), LY2157299 (S2230) and cisplatin (S1166) were purchased from Selleck Chemicals. 5-FU was obtained from the hospital pharmacy at The Netherlands Cancer Institute. Recombinant hTGFbeta 1 (240-B/CF) was purchased from R&D systems. TRC human genome-wide shRNA collection (TRC-Hs1.0) was purchased from Open Biosystems (Huntsville, USA). Further information is available at www.broad.mit.edu/genome_bio/trc/rnai.html.

Antibody against MED12 (A300-774A) and MED13 (A301-278A) was from Bethyl Laboratories; antibodies against Vimentin (RV202) and N-cadherin (ab18203) were from Abcam; antibody against p-SMAD2 (Ser465/467, #3101), SMAD2 (L16D3, #3103), pMEK1/2 (S217/221, #9121) and MEK1/2 (L38C12, #4694) were from Cell Signaling; anti-Flag M2 (F1804) was from Sigma; antibodies against HSP90 (H-114), p-ERK (E-4), ERK1 (C-16), ERK2 (C-14), CDK8 (D-9), Lamin A/C (636), SP1 (PEP2), alpha-TUBULIN (H-183), TGFbeta R2 (C-16), CDK8 (D9) and normal rabbit IgG control were from Santa Cruz Biotechnology; A mixture of ERK1 and ERK2 antibodies was used for detection of total ERK; 12CA5 hybridoma supernatant was also used to detect HA tagged-TGFbeta R2.

Endo H (P0702) and PNGase F (P0704) were purchased from New England Biolabs and used according to the manufacture's protocols.

H3122, PC9, H3255, SK-CO-1 and SW1417 cells were cultured in RPMI with 8% heat inactivated fetal bovine serum, penicillin and streptomycin at 5% CO2. HEK 293T, Phoenix and A375, SK-MEL-28 and Huh-7 cells were cultured in DMEM with 8% heat-inactivated fetal bovine serum, penicillin and streptomycin at 5% CO2. Subclones of each cell line expressing the murine ecotropic receptor were generated and used for all experiments shown. Phoenix cells were used as producers of retroviral supernatants as described at www.stanford.edu/group/nolan/retroviral_systems/phx.html. HEK 293T cells were used as producers of lentiviral supernatants as described at www.broadinstitute.org/rnai/public/resources/protocols. The calcium phosphate method was used for the transfection of Phoenix and 293T cells. Infected cells were selected for successful retroviral integration using 2 μg/ml of puromycin.

For long-term cell proliferation assays, cells were seeded into 6-well plates (2-5×10E4 cells/well) and cultured both in the absence and presence of drugs as indicated. More details are described (Huang et al., 2009. Cancer Cell 15: 328-340). All knockdown and overexpression experiments were done by retroviral or lentiviral infection. All relevant assays were performed independently at least three times.

All retroviral shRNA vectors were generated by ligating synthetic oligonucleotides (Invitrogen) against the target genes into the pRetroSuper (pRS) retroviral vector as described (Brummelkamp et al., 2002. Science 296, 550-553.) The following RNAi target sequences were used for retroviral shRNA vectors for this study:

shGFP:
GCTGACCCTGAAGTTCATC;
shMED12#1:
GTACCATGACTCCAATGAG;
shMED12#2:
GGAAGAGGTGTTTGGGTAC;
shMED12#3:
GGAGGAACTGCTTGTGCAC.

lentiviral shRNA vector shMED13#2 was generated as described at www.broadinstitute.org/rnai using the following target sequence: GCTCCAGACAGTCAAGTGAGA. All other lentiviral shRNA vectors were retrieved from the arrayed TRC human genome-wide shRNA collection (TRC-Hs1.0). Additional information about the shRNA vectors can be found at www.broadinstitute.org/rnai/public/clone/search using the TRCN number. The following lentiviral shRNA vectors were used:

shMED12#4: TRCN0000018576;
shMED12#5: TRCN0000018578;
shTGFbeta R2#1: TRCN0000000830;
shTGFbeta R2#2: TRCN0000010445;
shCDK8#1: TRCN0000000491;
shCDK8#2: TRCN0000000493;
shMED13#1: TRCN0000019916;
shSMURF#1: TRCN0000003475;
shSMURF#2: TRCN0000003476;
shSMURF#3: TRCN0000003478;
shSMURF#4, TRCN0000003477;
shSMURF#5: TRCN0000010792;
shSMAD7#1: TRCN0000019387;
shSMAD7#2: TRCN0000019384;
shSMAD7#3: TRCN0000019385;
shSMAD7#4: TRCN0000019386; and
shSMAD7#5: TRCN0000019388.

The details for the lentiviral shRNA vectors of the Custom TRC Kinome Library are described below.

The mouse Med12 expression constructs were generated by the following steps:

1) A linker containing first 89 bp of Med12 open reading frame (ORF) and multiple restriction sites was cloned into pcDNA3.1(+) vector by NheI and BamHI restriction sites and was sequence verified. The oligonucleotide sequences of the top strand for the linker was: 5′-CTAGCTCGAGTCGACCATGGCGGCTTT CGGGATCTTGAGCTATGAACACCGACCCCTGAAGCGGCTGCGGCTGGGGCCTC CCGATGTGTACCCTCAG and the bottom strand was: 5′-GATCCTGAGGGTA CACATCGGGAGGCCCCAGCCGCAGCCGCTTCAGGGGTCGGTGTTCATAGCTCA AGATCCCGAAAGCCGCCATGGTCGACTCGAG.
2) A PCR fragment of partial Med12 (from 89 to 1777 bp) was generated using a forward primer (5′-CAGGATCCCAAACAGAAGGAGGATGAACTGACGGCTTTGAATGTAA), a reverse primer (5′-TGGGAGAAGACATCATGTCG) and a Med12 partial cDNA as the template (IMAGE id: 6830443). The PCR fragment was then cloned into the pcDNA3.1(+)-Med12 (first 89 bp) vector described in step 1 by BamHI and EcoRI restriction sites and was sequence verified. Note that a silence mutation (A to G) at 81 bp of Med12 ORF was introduced in the forward PCR primer to generate BamHI site in the PCR fragment.
3) An EcoRI/NotI fragment (containing from 1778 to 6573 bp of Med12 ORF) from the Med12 partial cDNA (IMAGE id: 6830443) was cloned into the pcDNA3.1(+)-Med12 (first 1777 bp) described above by EcoRI and NotI restriction sites to generate the pcDNA3.1(+)-Med12 (full-length).
4) The XhoI/NotI fragment containing the full-length Med12 ORF from pcDNA3.1(+)-Med12 was then cloned into the retroviral expression vector pMX-IRES-blasticidine using the XhoI and NotI restriction sites.

The Flag-Med12 expression construct was generated by cloning annealed oligos containing in frame 3X Flag sequences into pMX-Med12 (described above) using the XhoI restriction site at 5″ of Med12 ORF and sequence verified. The oligos containing 3X Flag sequences are: XhoIKozac-3Xflag-SalI_Top: 5′-TCGAGACCATGGATTACAAGGATGACGACGATAAGGATTACAAGGATGACGAC GATAAGGATTACAAGGATGACGACGATAAGG; XhoIKozac-3Xflag-SalI_bottom: 5′-TCGACCTTATCGTCGTCATCCTTGTAATCCTTATCGTCGTCATCCTTG TAATCCTTATCGTCGTCATCCTTGTAATCCATGGTC.

Retroviral expression constructs (pBabe) for KRASG12V (#12544), MEK-DD (#15268), RALAQ75L (#19719), RALBQ72L (#19721), PIK3CAH1047R (#12524), pCMV5BTGFbeta R2 (#24801) and pQCXIP-TGFbeta R2-HA (#19147) were obtained from Addgene (Cambridge, Mass.) and sequence validated. The pBabe-BRAFV600E plasmid (Addgene 15269) was a kind gift of Daniel Peeper. The cDNA encoding Myr-AKT was cloned into pBabe-puro and validated by sequencing. These active alleles of RAS effector pathways were also described previously ((Brummelkamp et al., 2006. Nat Chem Biol 2, 202-206; Holzel et al., 2010. Cell 142, 218-229).

### Example 2

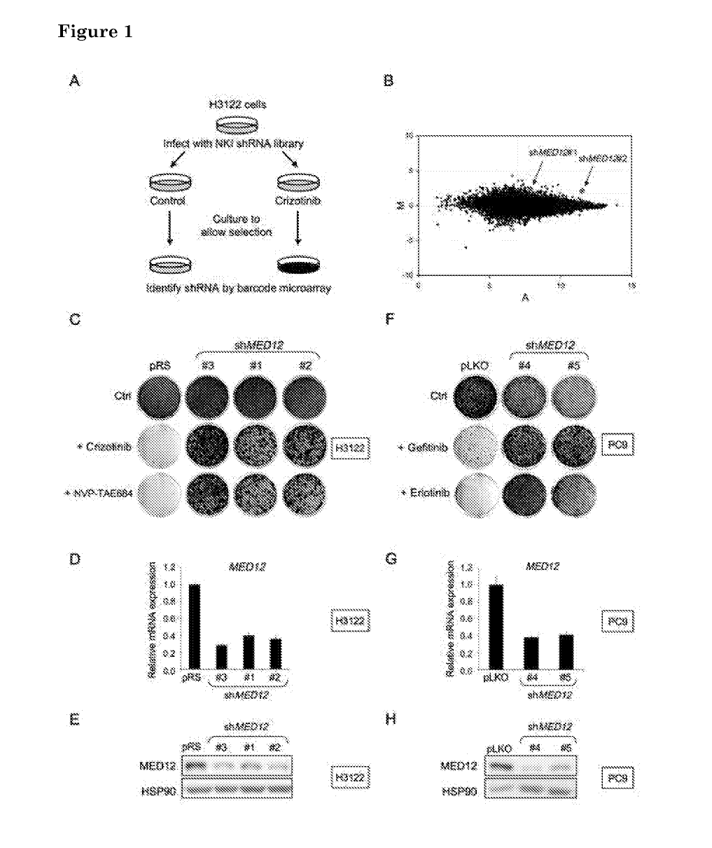

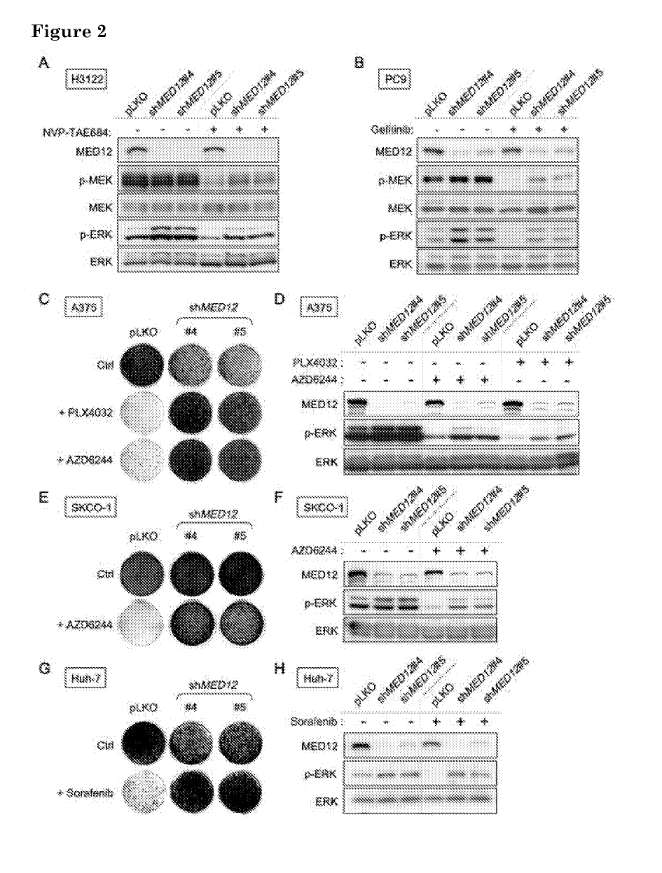

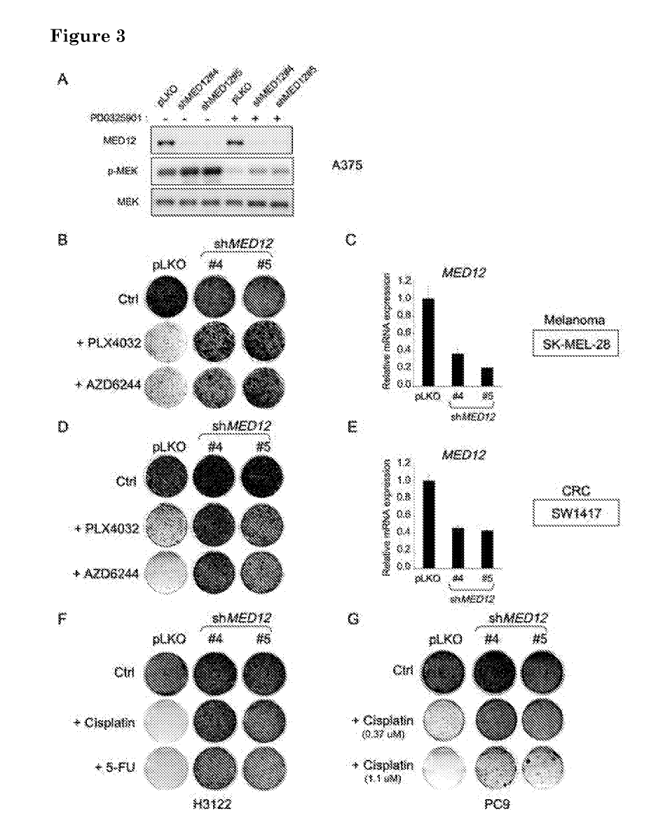

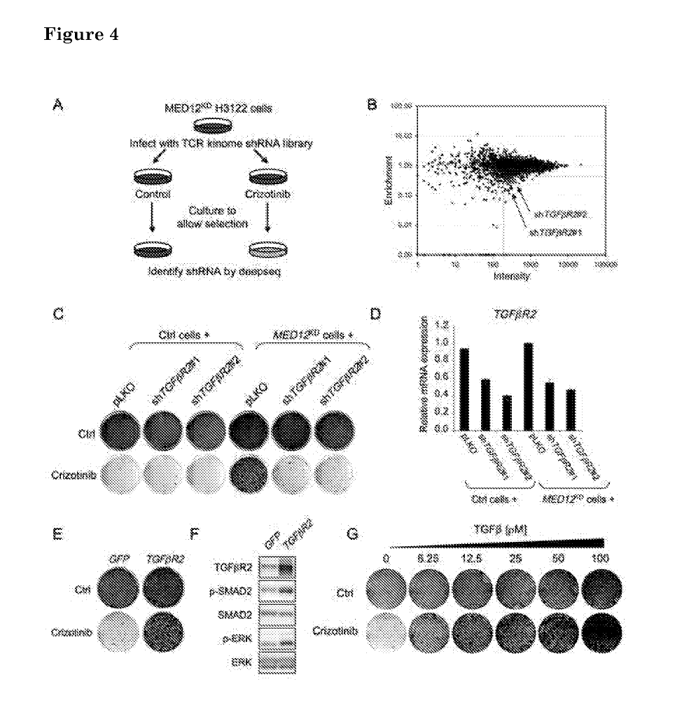

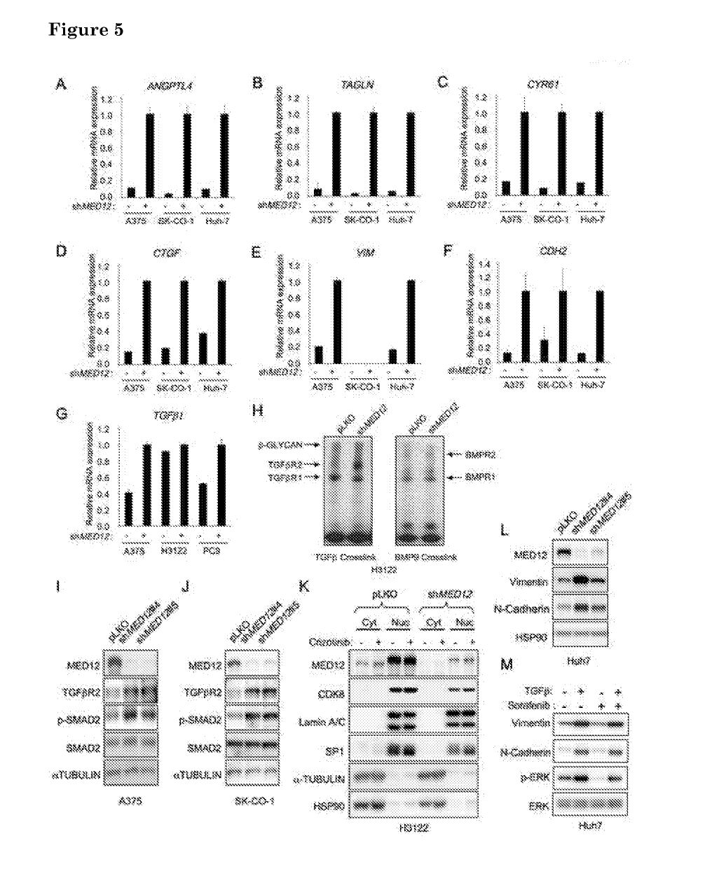

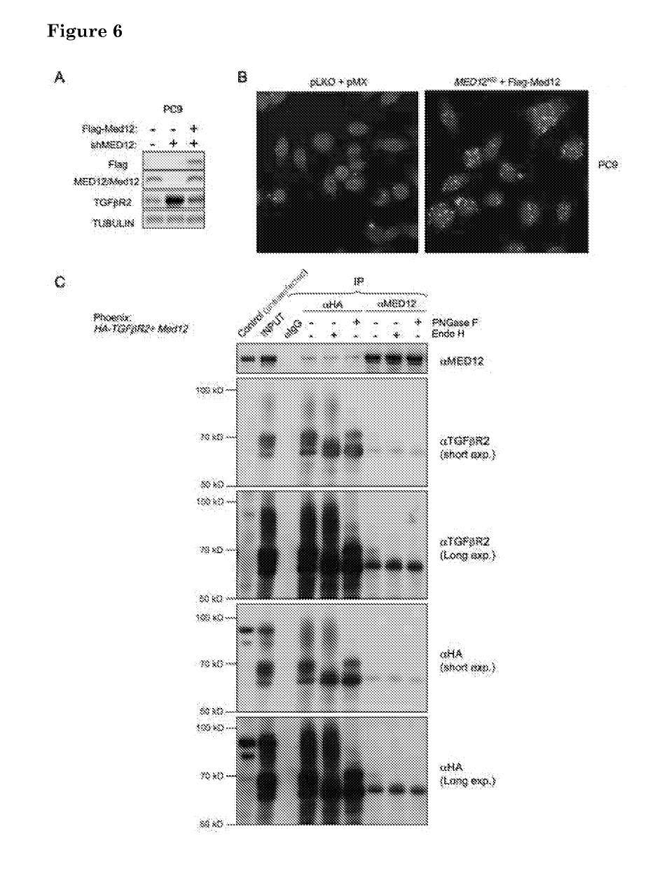

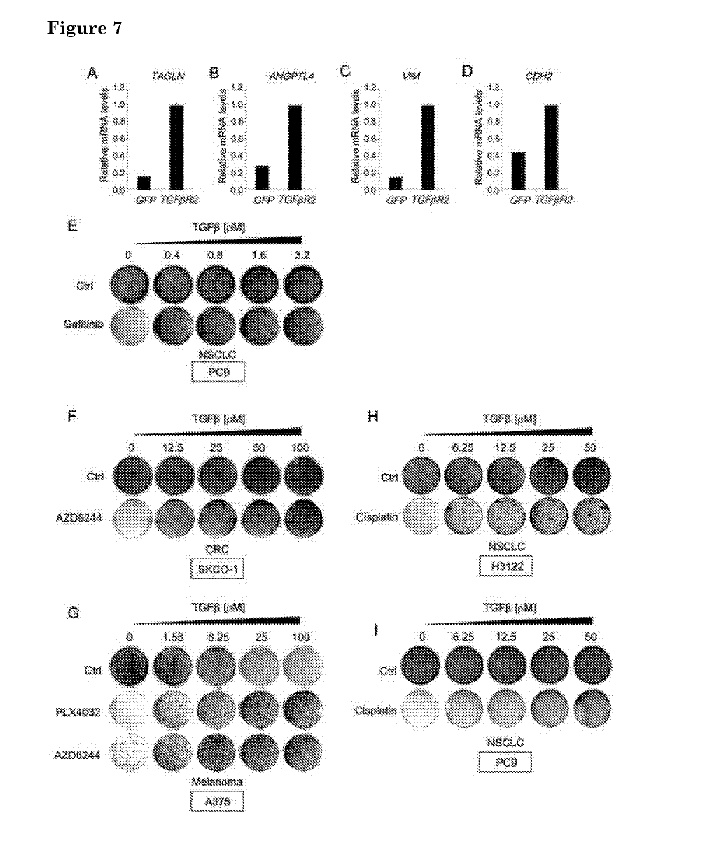

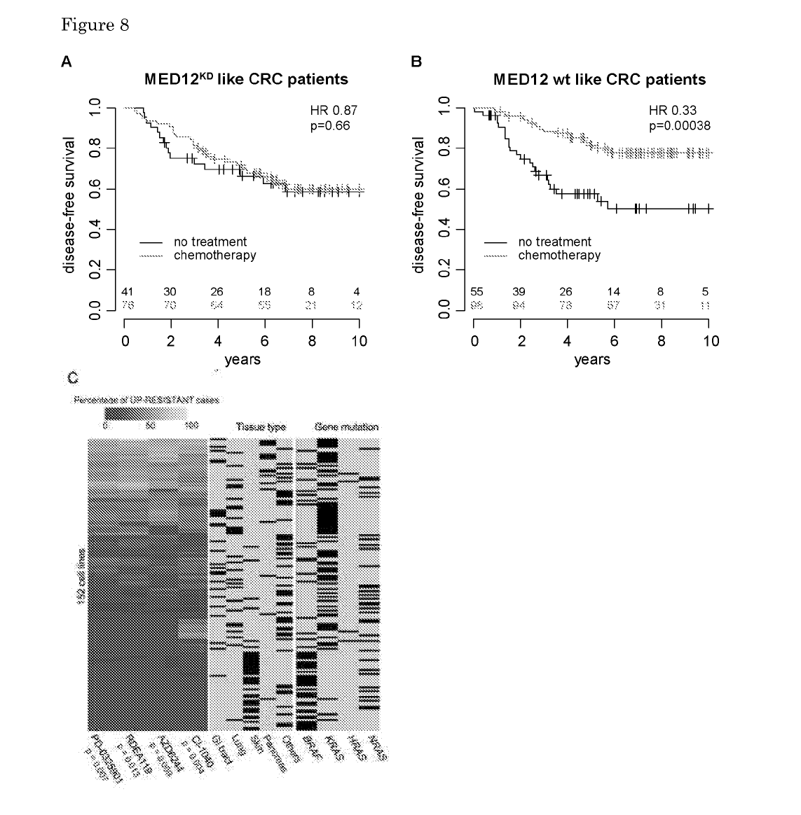

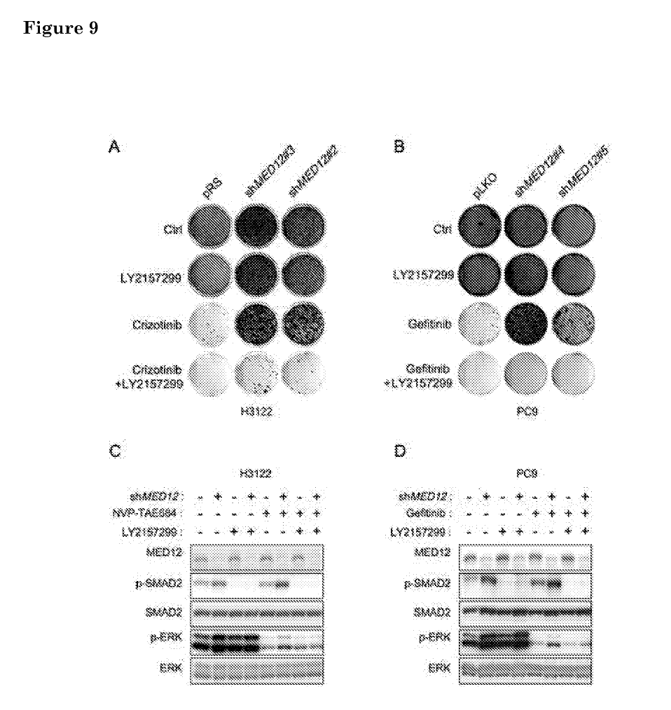

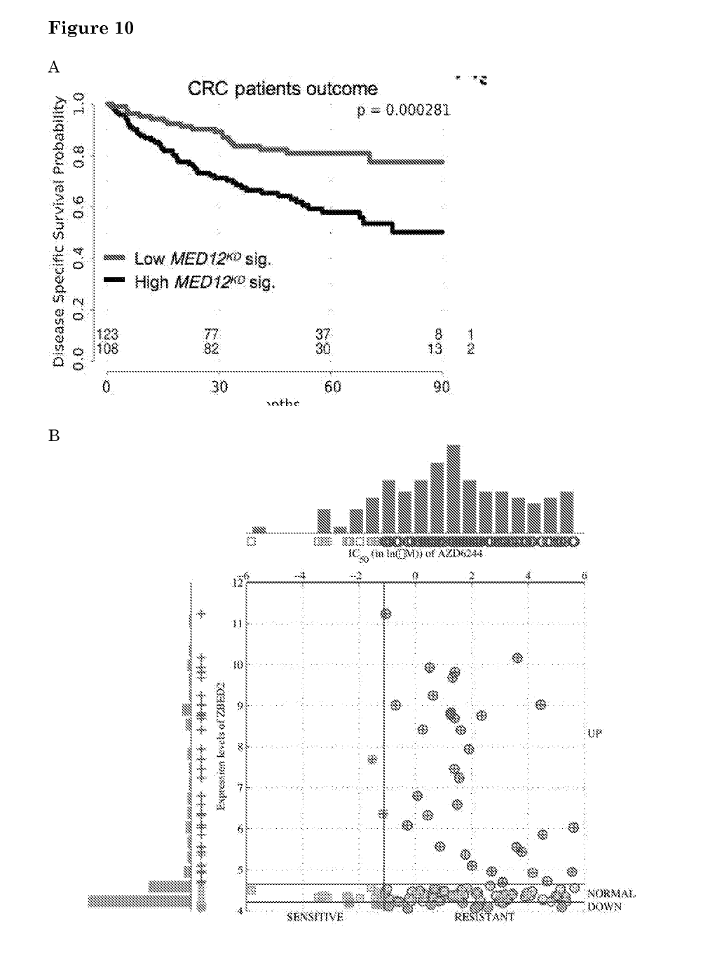

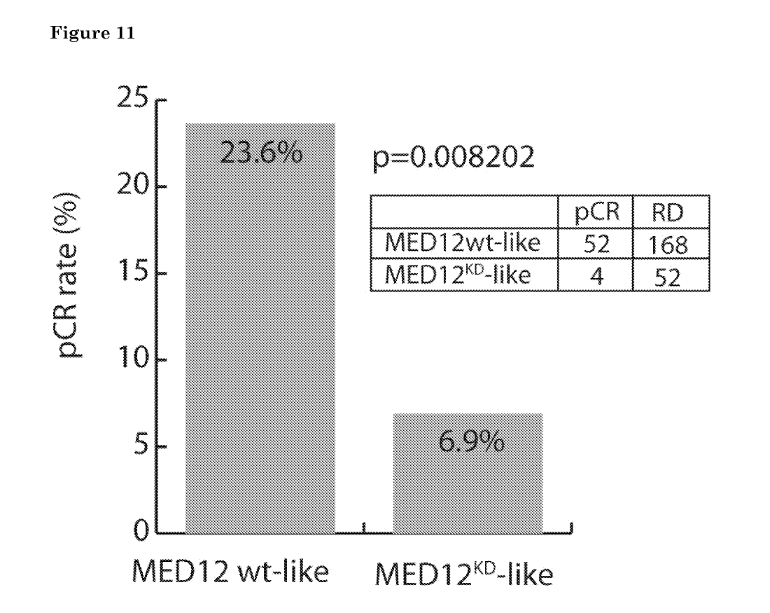

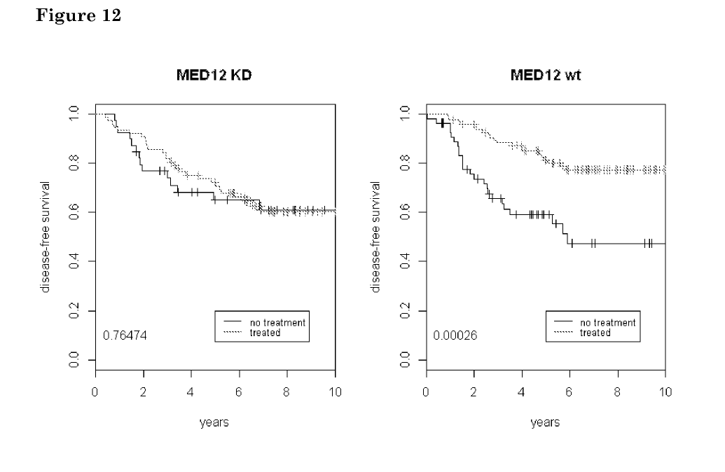

The NSCLC cell line H3122 harbors an EML4-ALK translocation and is exquisitely sensitive to the selective ALK inhibitors PF-02341066 (crizotinib) and NVP-TAE684 (McDermott et al., 2008. Cancer Res 68, 3389-3395). To identify genetic determinants of resistance to ALK inhibitors in EML4-ALK translocated NSCLC, we performed a large-scale RNAi-based loss-of-function genetic screen using a collection of 24,000 short hairpin (shRNA) vectors targeting 8,000 human genes (Berns et al., 2004. Nature 428, 431-437; Brummelkamp et al., 2002. Science 296, 550-553). As outlined in FIG. 1A, we used a barcoding technology to identify genes whose suppression causes resistance to crizotinib in H3122 cells (Brummelkamp et al., 2006. Nat Chem Biol 2, 202-206; Holzel et al., 2010. Cell 142, 218-229). The barcode screen results are shown in FIG. 1B. Each dot in the M/A-plot represents one individual shRNA vector. Low-intensity spots are prone to technical artifacts and thus unreliable. Therefore we restricted our candidate selection by applying M/A cut-off values as indicated in FIG. 1B. To rule out “off-target” effects, we only considered a gene identified in the screen as a genuine hit, if at least two independent shRNAs suppress the expression of the target and also confer crizotinib resistance. Only one gene fulfilled these criteria: MED12, encoding a component of the large MEDIATOR transcriptional adapter complex.

### Example 3

To validate MED12 as a gene whose suppression confers resistance to crizotinib, we individually introduced the two MED12 shRNA vectors (#1 and #2) from the library and one newly generated shRNA (#3) into H3122 cells by retroviral infection. Empty vector (pRS) or shRNA targeting GFP (shGFP) served as controls. All three distinct MED12 knockdown vectors conferred resistance to both crizotinib and NVP-TAE684 in colony formation assays (FIG. 1C) and also efficiently suppressed MED12 mRNA and protein expression (FIG. 1D, E). Similarly, expression of additional independent lentiviral shMED12 vectors (#4 and #5) in H3122 cells also conferred resistance to ALK inhibitors (data not shown). Furthermore, reconstitution of the RNAi-resistant murine Med12 cDNA in MED12 knockdown (MED12KD) H3122 cells restored the sensitivity of these cells to ALK inhibition (data not shown). Reconstituted Med12 protein in MED12KD cells was at a level comparable to that of parental cells (data not shown). Suppression of MED12 also conferred resistance to the EGFR inhibitors gefitinib or erlotinib in the EGFR mutant NSCLC cell lines PC9 and H3255 (FIG. 1F-H and data not shown). Together, these results establish a potential role for MED12 in resistance to ALK and EGFR inhibitor

### Example 4

Our finding that MED12 suppression confers resistance to both ALK and EGFR inhibitors in NSCLCs suggests that MED12 might act on a critical pathway downstream of both ALK and EGFR. RAS signaling is downstream of all activated RTKs in NSCLC (Pao and Chmielecki, 2010. Nature reviews 10, 760-774). We therefore asked whether the activity of the RAF-MEK-ERK pathway is altered in MED12KD cells. Indeed, H3122 cells expressing shMED12 vectors maintained higher levels of phosphorylated MEK (p-MEK) and ERK (p-ERK) in the presence of ALK inhibitor (FIG. 2A). Similarly, knockdown of MED12 in PC9 and H3255 cells leads to higher levels of p-MEK and p-ERK in both absence and presence of EGFR inhibitors (FIG. 2B, and data not shown). These findings suggest that MED12 loss confers resistance to ALK and EGFR inhibitors in NSCLCs by enhancing MEK/ERK activation.

### Example 5

Since MED12 suppression leads to ERK activation, one would expect that MED12 loss might also confer resistance to other cancer drugs targeting the kinases upstream of ERK. The small molecule drug PLX4032 (vemurafenib) is very effective in the treatment of BRAFV600E melanoma and the MEK inhibitor AZD6244 (seluteminib) is being tested for the treatment of several cancers. A375 melanoma cells (having BRAFV600E) are highly sensitive to PLX4032 and AZD6244. Consistent with our observations made in NSCLC models, we found that suppression of MED12 in A375 cells caused MEK/ERK activation (FIGS. 3A and 2D) and conferred potent resistance to both PLX4032 and AZD6244 (FIG. 2C). Similar results were obtained in the BRAFV600E melanoma cell line SK-MEL-28 (FIGS. 3B and 3C). SK-CO-1 colorectal cancer (CRC) cells harbor a KRASV12 mutation and are highly sensitive to MEK inhibition by AZD6244. MED12KD also resulted in activation of MEK/ERK (FIG. 2F and data not shown) and conferred resistance to AZD6244 in these cells (FIG. 2E). Identical results were observed in the CRC cell line SW1417 harboring a BRAFV600E mutation (FIGS. 3D and 3E).

### Example 6

To extend our findings, we asked whether MED12KD also confers resistance to a class of multi-kinase inhibitors. Sorafenib targets multiple kinases and is used clinically to treat renal cell carcinoma and hepatocellular carcinoma (HCC). Huh-7 HCC cells became resistant to sorafenib after knockdown of MED12 (FIG. 2G, H). In addition to targeted agents, MED12 knockdown also conferred resistance to chemotherapy drugs such as cisplatin and 5-Fluorouracil (5-FU) (FIGS. 3F, 3G). We conclude that the effects of MED12 suppression are mostly context-independent as its consequences are readily apparent in several major cancer types.

### Example 7

### shRNA “Dropout” Screen with a Custom TRC Kinome Library

A Kinome shRNA library targeting the full complement of 518 human kinases and 17 kinase-related genes was constructed from the TRC human genome-wide shRNA collection (TRCHs1.0). The Kinome library was used to generate pools of lentiviral shRNA to infect H3122 cells stably expressing shMED12. Cells were cultured in the presence or absence of crizotinib. Massive parallel sequencing was applied to determine the abundance of shRNA in cells. shRNAs prioritized for further analysis were selected by the fold of depletion by crizotinib treatment.

The kinome library consisted of 7 plasmids pools (TK1-TK7). Lentiviral supernatants were generated as described at www.broadinstitute.org/rnai/public/resources/protocols. H3122 cells stably expressing shMED12#3 were infected separately by the 7 virus pools (Multiplicity Of Infection of 1). Cells were then pooled and plated at 300,000 cells per 15 cm dish in absence or presence of 300 nM crizotinib (5 dishes for each condition) and the medium was refreshed twice per week for 10 days. Genomic DNA was isolated as described (Brummelkamp et al., 2006. Nat Chem Biol 2, 202-206). shRNA inserts were retrieved from Bug genomic DNA by PCR amplification (PCR1 and PCR2, see below for primer information) using the following conditions: (1) 98° C., 30 s; (2) 98° C., 10 s; (3) 60° C., 20 s; (4) 72° C., 1 min; (5) to step 2, 15 cycles; (6) 72° C., 5 min; (7) 4° C. Indexes and adaptors for deep sequencing (Illumina) were incorporated into PCR primers. 2.5 ul PCR1 products were used as templates for PCR2 reaction. PCR products were purified using Qiagen PCR purification Kit according to the manufacturer manual. Sample quantification was performed by BioAnalyzer to ensure samples generated at different conditions were pooled at the same molar ratio before analyzed by Illumina genome analyzer. shRNA stem sequence was segregated from each sequencing reads and aligned to TRC library. The matched reads were counted and the counts were transformed to abundance that was assigned to the corresponding shRNA.

Primers used were as follows:

PCR1_Untreated replicate#1_Forward:
5′-ACACTCTTTCCCTACACGACGCTCTTCCGATCTCTGATCCTTGTGGA
AAGGACGAAACACCGG;
PCR1_Untreated replicate#2_Forward:
5′-ACACTCTTTCCCTACACGACGCTCTTCCGATCTAAGCTACTTGTGGA
AAGGACGAAACACCGG;
PCR1_PLX treated replicate#1_Forward:
5′-ACACTCTTTCCCTACACGACGCTCTTCCGATCTGTAGCCCTTGTGGA
AAGGACGAAACACCGG;
PCR1_PLX treated replicate#1_Forward:
5′-ACACTCTTTCCCTACACGACGCTCTTCCGATCTTACAAGCTTGTGGA
AAGGACGAAACACCGG;
PCR1_Reverse (P7_pLKO1_r):
5′-CAAGCAGAAGACGGCATACGAGATTTCTTTCCCCTGCACTGTACCC;
PCR2_Forward:
5′-AATGATACGGCGACCACCGAGATCTACACTCTTTCCCTACACGACGC
TCTTCCGATCT:
PCR2_Reverse (P5_IlluSeq):
5′-CAAGCAGAAGACGGCATACGAGAT.

While the studies above showed that suppression of MED12 leads to ERK activation and thus confers what appears to be a “multi drug resistance” phenotype, they did not reveal through which pathway MED12 acts to mediate these effects. To gain further mechanistic insights, we set out to screen a lentiviral shRNA library representing all 518 human kinases (the “kinome”, (Manning et al., 2002. Science 298, 1912-1934)) and 17 kinase-related genes for genes whose inhibition restores sensitivity to ALK inhibitors in MED12KD cells. This “drop out” screen (FIG. 4A) is the inverse of the resistance screen shown in FIGS. 1A and 1B, as here we select for shRNAs that are depleted upon drug treatment rather than enriched. Among the top 51 candidates that met the selection criterion described in FIG. 4B, only one gene, transforming growth factor beta receptor II (TGFbeta R2), was represented by two independent shRNA vectors. This suggests that suppression of TGFbeta R2 synergizes with ALK inhibition in MED12KD cells. To validate this finding, we infected the same MED12KD H3122 cells with each of these two shTGFbeta R2 vectors (both of which reduced TGFbeta R2 levels (FIG. 4D)) and cultured these cells with or without crizotinib for two weeks. Inhibition of TGFbeta R2 did not significantly affect proliferation of the parental or MED12KD cells in the absence of crizotinib (FIG. 4C). In contrast, suppression of TGFbeta R2 in combination with ALK inhibitor caused a marked inhibition of proliferation in MED12KD cells (FIG. 4C). These findings indicate that suppression of TGFbeta R2 re-sensitizes the MED12KD cells to ALK inhibition and suggest that TGFbeta signaling is required for the drug resistance caused by MED12 loss.

### Example 8

Next, we asked whether TGFbeta signaling alone is sufficient to cause resistance to the cancer drugs studied above. We found that overexpression of exogenous TGFbeta R2 was sufficient to activate TGFbeta signaling (FIGS. 4F and 7A-D) and confer resistance to crizotinib in H3122 cells (FIG. 4E). Consistently, recombinant TGFbeta treatment also caused resistance to crizotinib in H3122 cells in a TGFbeta-dosage dependent manner (FIG. 4G). Furthermore, TGFbeta treatment also caused MEK/ERK activation in H3122 cells, consistent with the established activity of TGFbeta in non-SMAD pathway signaling (Zhang, 2009. Cell Res 19: 128-139; data not shown). These data indicate that TGFbeta activation, similar to suppression of MED12, is sufficient to confer resistant to ALK inhibitors in EML4-ALK positive NSCLCs. Recombinant TGFbeta treatment also caused MEK/ERK activation and conferred resistance to EGFR inhibitors in PC9 and H3255 NSCLC cells in a dosage-dependent manner (FIG. 7E, and data not shown). Similarly, TGFbeta-induced resistance to AZD6244 and PLX4032 was also observed in SK-CO-1 CRC cells and A375 melanoma cells (FIGS. 7F, 7G and data not shown). Finally, TGFbeta treatment also conferred resistance to cisplatin in H3122 and PC9 cells (FIG. 7H, I). In some cells, such as A375 and Huh-7 cells, (FIG. 7G and data not shown), recombinant TGFbeta treatment alone resulted in growth inhibition, but clearly became beneficial for proliferation when cells were cultured in the presence of targeted cancer drugs, mimicking the effects of MED12KD in the same cells (FIG. 2C, G). Collectively, these results demonstrate that activation of TGFbeta signaling is sufficient to confer resistance to multiple cancer drugs in the cancer types in which MED12KD also confers drug resistance.

### Example 9

Our findings that TGFbeta signaling is required for the drug resistance caused by MED12KD and that activation of TGFbeta signaling phenocopies MED12KD in causing drug resistance suggested that MED12 can act as a suppressor of TGFbeta signaling. We explored this by studying gene expression analysis using next generation sequencing (RNA-Seq) in a panel of cells lines (H3122, PC9, SK-CO-1, A375 and Huh-7), and multiple MED12KD derivatives thereof. Many of the genes of which the level of expression was modulated by MED12KD were bona fide TGFbeta targets. To confirm these observations, we first examined mRNA expression levels of a panel of TGFbeta target genes, including ANGPTL4, TAGLN, CYR61, CTGF, SERPINE1 and CDKN1A in both H3122 and PC9 cells by qRT-PCR (FIG. 7A-D and data not shown). In agreement with our RNA-Seq data, all of these TGFbeta target genes were significantly induced upon MED12KD in these NSCLC cells. We also observed induction of TGFbeta mRNA expression in A375 and PC9 cells upon MED12KD (FIG. 5G) and of the TGFbeta target genes upon MED12KD in other cancer types, including melanoma, colon cancer and HCC (FIG. 5A-D and data not shown). It is well-established that TGFbeta induces an epithelial-mesenchymal transition (EMT), leading to the induction of several mesenchymal markers such as Vimentin (VIM) and N-cadherin (CDH2) (Thiery et al., 2009. Cell 139, 871-890). MED12KD also induced expression of VIM and CDH2, indicating that an EMT-like process is initiated in MED12KD cells (FIG. 5E-F and data not shown). Accordingly, the protein products of these mesenchymal-specific genes were also detected in MED12KD cells (FIG. 5L and data not shown), similar to the levels induced by treatment of TGFbeta in the same cells (FIG. 5M). Expression of the epithelial marker E-cadherin (CDH1) was not lost in MED12KD cells (data not shown), suggesting that MED12KD induces a partial EMT. Together, these unbiased gene expression studies support the notion that MED12 is a suppressor of TGFbeta signaling in a wide range of cancer types and that its loss activates TGFbeta signaling.

### Example 10

### M&M Quantitative RT-PCR (qRT-PCR)

QRT-PCR assays were carried out to measure mRNA levels of genes using 7500 Fast Real-Time PCR System (Applied Biosystems) as described (Kortlever et al., 2006. Nat Cell Biol 8, 877-884). Relative mRNA levels of each gene shown were normalized to the expression of the house keeping gene GAPDH. The sequences of the primers for assays using SYBR® Green master mix (Roche) were as follows:

GAPDH_Forward:
AAGGTGAAGGTCGGAGTCAA;
GAPDH_Reverse:
AATGAAGGGGTCATTGATGG;
MED12_Forward:
GCTGGTGCACATAGCCACT;
MED12_Reverse:
TACTCCAGCCAGCCTTACCA;
Med12_Forward:
TCAGGCAGTGGGATTACAATGA;
Med12_Reverse:
TCCAGGGCGTATTTTCTCAAAAC;
TGFbeta R2_Forward:
GCACGTTCAGAAGTCGGTTA;
TGFbeta R2_Reverse:
TCTGGTTGTCACAGGTGGAA;
ANGPTL4_Forward:
GGAACAGCTCCTGGCAATC;
ANGPTL4_Reverse:
GCACCTAGACCATGAGGTGG;
TAGLN_Forward,
GTCCGAACCCAGACACAAGT;
TAGLN_Reverse:
CTCATGCCATAGGAAGGACC;
CYR61_Forward:
GCTGGAATGCAACTTCGG;
CYR61_Reverse:
CCCGTTTTGGTAGATTCTGG;
CTGF_Forward:
TACCAATGACAACGCCTCCT;
CTGF_Reverse:
TGGAGATTTTGGGAGTACGG;
VIM_Forward:
CTTCAGAGAGAGGAAGCCGA;
VIM_Reverse:
ATTCCACTTTGCGTTCAAGG;
CDH2_Forward:
CCACCTTAAAATCTGCAGGC;
CDH2_Reverse:
GTGCATGAAGGACAGCCTCT;
TGFbeta 1_Forward:
CCCTGGACACCAACTATTGC;
TGFbeta 1_Reverse:
CTTCCAGCCGAGGTCCTT.

To further elucidate the molecular mechanism by which MED12 suppresses TGFbeta signaling, we studied the effect of knockdown of MED12 on expression and activation of key components of the TGFbeta signaling pathway. We found that suppression of MED12 resulted in a strong induction of TGFbeta R2 protein levels in H3122 and PC9 cells (data not shown). As a result of the TGFbeta R2 upregulation, SMAD2, the key mediator for TGFbeta target gene activation, was activated as indicated by a strong increase in SMAD2 phosphorylation upon MED12 knockdown. Consistently, affinity-labeling assays using 125I-TGFbeta 1 showed strong increase of the 125I-labeled cell surface TGFbeta R2 upon MED12KD in H3122 cells (FIG. 5H). As controls, 125I-BMP9 affinity-labeling experiments showed no significant change in labeled BMP receptors upon MED12KD. Similar results were obtained in A375 melanoma and in SKCO-1 CRC cells, indicating that this interplay between MED12KD and TGFbeta signaling is conserved across different cancer types (FIG. 5I-J). In conclusion, the upregulation of TGFbeta R2 in MED12KD cells causes the activation of TGFbeta signaling which in turns leads to MEK/ERK activation. Supporting this notion, downregulation of TGFbeta R2 by RNAi suppressed the MEK/ERK activation in MED12KD cells (data not shown).

### Example 11

### M&M Affinity Labeling of Cell Surface Receptors for TGFbeta and BMP9

Iodination of TGFbeta and BMP-9 was performed according to the chloramine T method (Frolik et al., 1984) and cells were subsequently affinity-labeled with the radioactive ligand as described before (Yamashita et al., 1995). In brief, cells were incubated on ice for 2 h with the radioactive ligand. After incubation, cells were washed and crosslinking was performed using 54 mM disuccinimidyl suberate (DSS) and 3 mM bis(sulfosuccinimidyl)suberate (BS3, Pierce) for 15 min. Cells were washed, scraped and lysed. Lysates boiled in sodium dodecyl sulphate (SDS) sample buffer and subjected to SDS-poly acrylamide gel electrophoresis (PAGE) directly or were incubated with anti-receptor antibodies overnight and immune complexes were precipitated by adding protein A Sepharose (Amersham). Samples were washed, boiled in SDS sample buffer and subjected to SDS-PAGE. Gels were dried and scanned with the STORM imaging system (Amersham).

### M&M Nuclear and Cytoplasmic Fractionation and Co-Immunoprecipitation

Subcellular fractionation experiments were performed according manufacture protocol using the NE-PER Nuclear and Cytoplasmic Extraction Kit (78835) purchased from Thermo Scientific. For the co-immunoprecipitation experiments using cytoplasmic fraction of PC9, 15-20 millions cells were used for fractionation assay per immunoprecipitation condition. The cytoplasmic fraction was dialyzed in buffer containing 200 mM NaCl, 50 mM Hepes KOH (PH7.6), 0.1% NP40, 0.2 mM PMSF and 0.5 mM Sodium metabisulfite for 6 hours before immunoprecipitation. For all co-immunoprecipitation experiments, the total lysates or cytoplasmic fractions were first pre-cleared with Protein A-sepharose beads before immunoprecipitation using rabbit IgG or the anti-MED12 antibody described above. 2% of input was loaded in the SDS-PAGE for the western blotting analysis.

### M&M Proximity Ligation Assay (PLA)

Duolink In Situ Proximity Ligation Assay® (PLA) detection kit was from Olink Bioscience and assays were performed according manufacture protocol. Final images were taken using a Leica SP5 (Live) confocal microscope system using a 60× oil objective lens.

Since MED12 is part of the MEDIATOR transcriptional complex that functions in the nucleus, we assumed that MED12 would act on TGFbeta R2 transcription. However, there was only a modest increase in TGFbeta R2 mRNA upon MED12 knockdown (data not shown). Moreover, we observed a progressive increase in TGFβR2 protein levels in time after MED12 knockdown. These results suggest that MED12 predominantly suppresses TGFβR2 in a posttranscriptional manner. To investigate this, we determined the subcellular localization of MED12. We carried out nuclear and cytoplasmic fractionation of PC9 cells expressing control vector or shMED12, followed by western blotting (data not shown). Lamin A/C and SP1 were used as controls for nuclear fractions, while alpha-TUBULIN and HSP90 were used as controls for cytoplasmic fractions. Abundant nuclear MED12 was detected, consistent with its function in the MEDIATOR transcriptional complex.

Unexpectedly, a significant quantity of MED12 was also present in the cytoplasmic fraction. Cytoplasmic MED12 was also seen in H3122 cells (FIG. 5K). Interestingly, no significant cytoplasmic CDK8 was detected, another subunit of the MEDIATOR kinase module with which MED12 is known to associate closely. This suggests that cytoplasmic MED12 might have a second function, distinct from its role in the MEDIATOR complex. Consistent with this, downregulation of other MEDIATOR subunits such as CDK8 and MED13 in PC9 and H3122 cells did not lead to upregulation of TGFbeta R2 or activation of SMAD2 (data not shown) and failed to confer resistance to EGFR and ALK inhibitors (data not shown).

The unexpected cytoplasmic localization of MED12 prompted us to examine a potential physical interaction between MED12 and TGFbeta R2. We first performed coimmunoprecipitation (Co-IP) experiments using Phoenix cells co-transfected with TGFbeta R2 and MED12. TGFbeta R2 co-immunoprecipitated with MED12 and conversely MED12 co-immunoprecipitated with TGFbeta R2, indicating that MED12 interacts physically with TGFbeta R2 (data not shown). Consistent with this, co-IP experiments using the cytoplasmic fraction of untransfected PC9 cells indicate that endogenous TGFbeta R2 interacts with endogenous MED12 (data not shown). As a second independent approach, we used a proximity ligation assay (PLA) to validate the TGFbeta R2-MED12 interaction in situ. PLA technology allows sensitive detection of protein-protein interaction, and requires two primary antibodies from different species against the proteins that are presumed to interact. Since our best antibodies against TGFbeta R2 and MED12 were produced in rabbits, we generated MED12KD PC9 cells reconstituted with Flag-Med12 to be able to use PLA technology using mouse anti-Flag to detect Med12. These reconstituted cells expressed similar levels of MED12 and TGFbeta R2 proteins as parental cells (FIG. 6A). The results shown in FIG. 6B indicate that there is a significant in situ interaction of TGFbeta R2 and MED12 in the cytoplasm of PC9 cells, which is consistent with the data from the Co-IP experiments above.

The observation that MED12KD caused a strong increase of cell surface TGFbeta R2 (FIG. 5H) suggests that MED12 could inhibit TGFbeta R signaling by preventing the maturation of TGFbeta R2. To test this hypothesis, we performed Co-IP experiments using antibodies against HA tag and MED12 on Phoenix cells co-transfected with HA-TGFbeta R2 and MED12, and incubated the immunoprecipitates with Endo H or PNGase F enzymes. Endo H selectively removes oligosaccharides of glycoproteins in endoplasmic reticulum (ER), but not the highly processed complex oligosaccharides processed in Golgi. In contrast, PNGase F deglycosylates glycoproteins in both ER and Golgi. As indicated in FIG. 6C, in the TGFbeta R2 immunoprecipitate, we observed three distinct forms of TGFbeta R2: the 60 kDa form that was insensitive to both Endo H and PNGase F, therefore presumably corresponding to the unglycosylated form of TGFbeta R2, the 70 kDa form that was sensitive to Endo H, corresponding to the partially glycosylated TGFbeta R2 in ER; the smear from 80 to 100 kDa that was Endo H-resistant but PNGase F-sensitive, corresponding to the fully glycosylated form of TGFbeta R2. We found that only the non-processed and partially processed ER (to a lesser extent) forms of TGFbeta R2 co-immunoprecipitated with MED12. These data are consistent with a model in which MED12 interferes with the proper glycosylation of TGFbeta R2 and hence blocks cell surface expression of the receptor (Kim et al., 2012. Biochemical Journal 445, 403-411).

### Example 12

Next, we asked if the MED12KD signature could also predict responses to chemotherapy in patients by examining of a second cohort of 270 stage III colorectal cancer patients, only part of which were treated with 5-FU-based chemotherapy and whose responses to chemotherapy are known (Maak et al., 2012. Ann Sur. In press; Salazar et al., 2011. J Clin Oncol 29, 17-24). Using the probes for MED12KD signature genes that were present on the microarray previously used for the expression analysis of these cancers (genes that are rank-ordered 1-46), patients were classified as MED12KD like or MED12 wild type (MED12 wt) like (see FIG. 8). We found that chemotherapy did not lead to noticeable change in disease-specific survival of patients with MED12KD like cancers (FIG. 8A), whereas it did cause a significant increase in disease-specific survival of patients with MED12 wt like cancers (FIG. 8B). These results indicate that the MED12KD signature predicts response to 5-FU-based chemotherapy in CRC patients.

### Example 13

### M&M RNA-Seq Gene Expression Analysis

Total mRNA of each sample was converted into a library of template molecules suitable for subsequent cluster generation using the reagents provided in the Illumina® TruSeq™ RNA Sample Preparation Kit, following the manufacture protocol. Sequence reads were generated using Illumina HiSeq 2000 with TruSeq™ v3 reagent kits and software. The reads (between 20-45 million 50 or 75 bp paired-end reads per sample) were mapped to the human reference genome (build 37) using TopHat (v. 1.3.1, (Trapnell et al., 2009)), which allows to span exon-exon splice junctions. The open-source tool HTSeq-count (v. 0.5.3p3), available from EMBL, was then used to generate a list of the total number of uniquely mapped reads (between 16-33 million pairs of reads per sample) for each gene that is present in the provided Gene Transfer Format (GTF) file.

M&M In order to determine which genes are differentially expressed between samples, the R package DEGseq (Wang et al., 2010. Bioinformatics 26, 136-138) was used, which takes the output of HTSeq-count as input. The method used to identify differentially expressed genes is the MA-plot-based method with technical Replicates (MATR), which makes use of the presence of technical replicates. The genes that have no expression for all samples in the comparison were discarded from the dataset. The expression levels of all remaining genes in the dataset were added with 1 in order to avoid negative values after log 2 transformation. Normalization for the number of reads is performed within this method and the cut off for differentially expressed genes is based on a p-value of 0.05.

To rule out “off-target” effects, we considered genes that are significantly deregulated in the same direction by two independent shMED12 vectors. The MED12KD gene signature was then assembled containing genes that were more than 2 fold up- or downregulated upon MED12 knock-down in at least three out of five cell lines. We employed next generation sequencing (RNA-seq) data from pairs of NSCLC cancer samples to determine genes that were upregulated after becoming resistant to gefitinib. Here, we describe how these upregulated gene sets were determined. For each pair of NSCLC cancer samples, genes with a normalized read count corresponding to a raw read count smaller than 10 in either of the two samples were excluded from this analysis to prevent artificially inflated log ratios. Genes were selected as upregulated after acquisition of gefitinib resistance if they were 2-fold upregulated in the sample after acquisition of gefitinib resistance compared to the sample before occurrence of the resistance. We employed the hypergeometric test to determine the significance in the overlap analyses.

### M&M Gene Expression Statistical Analysis

A) Prognostic value of a MED12KD signature Gene expression datasets GSE14333 (Jorissen et al., 2009. Clin Cancer Res 15, 7642-7651), GSE17536 and GSE17537 (Smith et al., 2010. Gastroenterology 138, 958-968) were downloaded from the Gene Expression Omnibus (Barrett et al., 2011. Nucleic Acids Res 39, D1005-1010). Duplicated samples in GSE14333 and GSE17536 were removed from GSE14333 resulting in a final dataset comprising 389 cancer samples. Disease specific survival (DSS) information was available for 231 of these 389 samples. Expression data were first normalized together using the RMA method as implemented in the affy package (Gautier et al., 2004. Bioinformatics 20, 307-315) for R/Bioconductor (Gentleman et al., 2004. Genome Biol 5, R80) and then mean-centered separately for each dataset. The hclust method was employed for hierarchically clustering the samples based on MED12KD and Pearson correlation distance. The survival and Design packages were used for performing a Kaplan-Meier survival time analysis and plotting survival curves, respectively.

B) Predictive value of a MED12KD signature

The presence of a MED12KD signature was associated with non responsiveness to chemotherapy on a panel of 270 stage III cancers from colorectal cancer patients (Maak et al., 2012. Ann Sur in press; Salazar et al., 2011. J Clin Oncol 29, 17-24). Of these patients, 174 received adjuvant 5FU-based chemotherapy and 96 received no chemotherapy. Readout of the MED12KD signature was performed using a subset (see Table 1) of the upregulated genes from MED12KD signature that were present on the custom made diagnostic microarray (Agilent), which was used previously for gene expression analysis. Following data quantification and normalization, as described previously (Maak et al., 2012. Ann Sur in press), cancer samples were classified as MED12 knockdown-like (MED12KD like) if at least 23 of the 46 genes were upregulated. All other cancers were classified as MED12 wildtype-like (MED12 wt like). Investigation of benefit from chemotherapy was performed by comparing the Kaplan-Meier (KM) disease-free survival curves of patients with and without chemotherapy.

### Results

To further substantiate our finding that MED12 suppression confers resistance to cancer drugs targeting the MEK-ERK pathway, we asked if the MED12KD signature could predict responses to MEK inhibitors in a large and heterogeneous panel of cancer cell lines of different tissue types. Since MEK inhibitors are currently being evaluated for the treatment of cancers having activating mutations in RAS or BRAF, we focused our studies on 152 cancer cell lines harboring either RAS or BRAF mutations for whom the IC50 values of four different MEK inhibitors and gene expression patterns have been determined (Garnett et al., 2012. Nature 483, 570-575). Of the 234 genes that were up-regulated by MED12KD as identified by next generation sequencing (RNA-Seq), we could read the expression levels for 170 genes in these 152 cell lines. We found that high expression of these 170 genes is significantly associated with higher IC50s for all four MEK inhibitors in these cell lines (AZD6244, p=0.009; CI-1040, p=0.004; PD-0325901, p=0.007; RDEA119, p=0.013; (FIG. 8C). Thus, the group of genes that is upregulated following MED12KD can predict response to MEK inhibitors in a very heterogeneous panel of cancer cell lines, consistent with the notion that MED12 acts independent of cellular context to influence cancer drug responses.

### Example 14

### M&M RNA Isolation from Patient Cancer FFPE Material

Cancer samples derived from three patients (case #3, 6 and 10) having NSCLC cancers with EGFR activating mutations both before and after acquisition of resistance to gefitinib are as described (Uramoto et al., 2011. Lung Cancer 73, 361-365). The institutional review board's approved informed consent for the use of the cancer tissue specimens was obtained either from all the patients or from the patient“s legal guardians.

RNA from FFPE material was extracted using the High Pure RNA paraffin kit (Roche), previously described (Bibikova et al., 2004. Am J Pathol 165, 1799-1807; Fan et al., 2004. Genome research 14, 878-885; Mittempergher et al., 2011. PLoS One 6, e17163). From the FFPE blocks, 4-5 sections of 3 μm were cut and put onto a microscope slide (1 section per slide). A 2-μm pre-cut section was stained with haematoxylin and eosin and reviewed by a pathologist to assess the cancer cell percentage. Only samples with cancer cell percentage at least equal to 60% were included in the following analysis. The isolation procedure was performed using minor adjustments of the manufacturer's instructions. Briefly, the glass slides were incubated at 75° C. for 10 minutes followed by 2 times 5 minutes in xylene. The sections were dissected by scratching off the enriched cancer cell area, using a sterile single-use scalpel and placed in a 1.5 ml reaction tube. To the tissue pellet deparaffinized as described above were added 100 μl tissue lysis buffer, 16% μl SDS and 40 μl Proteinase K working solution followed by incubation overnight at 55° C. The RNA isolation continued according to the manufacturer“s instructions. The RNA concentration was measured using the NanoDrop™ 2000 (Thermo scientific).

### Results

We asked whether expression of MED12KD signature genes is associated with drug resistance to targeted agents in the clinic. Pairs of cancer samples derived from three patients (case 3, 6 and 10) having NSCLC cancers with EGFR activating mutations were obtained from both before and after development of resistance to gefitinib (Uramoto et al., 2011. Lung Cancer 73, 361-365). Two of the resistant cancers did have the EGFR T790M gatekeeper mutation (case 3 and 6). RNA was isolated from these formalin-fixed cancer slides followed by transcriptome sequencing using Whole Transcriptome Shotgun Sequencing. For each pair, we selected genes that show a greater than 2-fold upregulation after acquisition of gefitinib resistance and then asked whether these genes overlap with the MED12KD signature. For the two cancer pairs with EGFR T790M mutations (case 3 and 6), we did not detect a significant overlap between MED12KD signature genes and the genes upregulated after gefitinib resistance (data not shown). For the cancer pair without the EGFR T790M mutation (case 10), we did observe a significant overlap of genes upregulated after acquisition of gefitinib resistance with the MED12KD signature genes. This result indicates that in this patient a gene expression program was activated upon gefitinib resistance that resembles the program induced by MED12KD.

### Example 15

Our data show that activation of TGFbeta by either MED12 loss or by recombinant TGFbeta confers resistance to multiple targeted cancer drugs in a variety of cancer types. It is therefore of potential clinical relevance to explore new treatment strategies to target drug resistant cancers having acquired elevated TGFbeta signaling. Since inhibition of TGFbeta R2 by RNAi resensitized MED12KD NSCLC cells to TKIs (FIG. 4), we reasoned that TGFbeta R inhibitors would synergize with TKIs to inhibit proliferation in MED12KD NSCLC cells.

To test this concept, we cultured both parental and MED12KD H3122 cells in the absence and the presence of crizotinib, the TGFbeta R inhibitor LY2157299 or the combination of both drugs (FIG. 9A). Consistent with our previous data, crizotinib alone potently inhibited the proliferation of the control, but not of the MED12KD cells. LY2157299 monotherapy had little effect on all cells. However, strong synergy was seen when crizotinib was combined with LY2157299, consistent with the notion derived from the RNAi experiment that TGFbeta R2 inhibition restored the sensitivity of MED12KD cells to crizotinib. Importantly, the same synergistic response was also obtained when LY2157299 was combined with gefitinib to suppress proliferation of MED12KD PC9 cells (FIG. 9B). Moreover, the combination of LY2157299 with crizotinib or gefitinib suppressed the ERK activation driven by MED12KD in both H3122 and PC9 cells (FIGS. 9C and 9D). These biochemical data are in line with our RNAi results where TGFbeta R2 knockdown suppressed ERK activation in MED12KD cells (data not shown). Thus, the combination of TGFbeta R inhibitors and TKIs might be a strategy for treating cancers with elevated TGFbeta signaling.

### Example 16

The presence of a MED12 knock down signature was associated with non responsiveness to chemotherapy on a panel of 267 previously characterized breast cancer patients [Iwamoto et al, J Clin Oncol. 2012 Mar. 1; 30(7):729-34]. These patients received taxane-based neoadjuvant chemotherapy (mainly TFAC regimen: paclitaxel, 5-fluorouracil, adriamycin, and cyclophosphamide). Readout of the MED12 gene signature was performed using 41 signature genes that were present on the Affymetrix platform (U133A). Following standard Affymetrix data quantification and normalization, the cancer samples were classified as MED12 knock-down-like (MED12KD-like) if at least 50 percent of the genes (21 of 41) were upregulated. All other cancers were classified as MED12 wildtype-like (MED12 wt-like). Patients classified as MED12KD-like showed a significant reduced response to taxane-based neoadjuvant chemotherapy with a pCR rate of 6.9% compared to a response rate of 23.6% for MED12 wt-like patients (p=0.0082) (FIG. 11).

### Example 17

Performance of the MED12 knock-down gene signature based on the 41-gene set, that was found to be significantly associated with pCR rates in breast cancer (example 16), was compared to the performance of the 46-gene set (see example 12) on prediction resistance to chemotherapy in colorectal cancer patients. The 41-gene signature showed an equal performance in prediction of benefit of chemotherapy on colorectal cancer patients. Patients classified as MED12 wildtype like showed a significant benefit of therapy compared to no response for MED12KD-like patients (FIG. 12).

TABLE 1

| | | Regulation | |
|---|---|---|---|
| | | upon | |
| | | MED12 | |
| Rank | | knock- | |
| No | Symbol | down | Sequence of probe |
| 1 | LGALS1 | up | ACTCAATCATGGCTTGTGGTCTGGTCGCCAGCAACCTGAATCTCAAACCTGGAGAGTGCC |
| 2 | EMP3 | up | TGGTACGACTGCACGTGGAACAACGACACCAAAACATGGGCCTGCAGTAATGTCAGCGAG |
| 3 | SPOCK1 | up | CTTTTTCTCAAAGTCACTGATGTTTGTTCCTGTTAAATGTATAGCATTGTAATGAGAGCC |
| 4 | TAGLN | up | TACCTTCAGCCCTGGCCAAGCTTTGAGGCTCTGTCACTGAGCAATGGTAACTGCACCTGG |
| 5 | CTGF | up | TGGAACTACATTAGTACACAGCACCAGAATGTATATTAAGGTGTGGCTTTAGGAGCAGTG |
| 6 | CDH2 | up | TGCCTCTGTATTGTGTACCAGAATATAAATGATACACCTCTGACCCCAGCGTTCTGAATA |
| 7 | TMEM45A | up | CTGAACGAGAACAAGAATCAGAAGAAGAAATGTGACTTTGATGAGCTTCCAGTTTTTCTA |
| 8 | TNC | up | TTTTACCAAAGCATCAATACAACCAGCCCAACCATCGGTCCACACCTGGGCATTTGGTGA |
| 9 | ECM1 | up | GTCAGATGGGGGGAACCCCACCCTGCCCCACCCATCTGAACACTCATTACACTAAACACC |
| 10 | DKK3 | up | AGAAAATCAAACCGAGCAGGGCTGTGTGAAACATGGTTGTAATATGCGACTGCGAACACT |
| 11 | MAP1B | up | TTGCAGTAATGATATTTATTAAAAACCCATAACTACCAGGAATAATGATACCTCCCACCC |
| 12 | MYLK | up | GGTAAATGAGAACACTACAACTGTAGTCAGCTCACAATTTTTAAATAAAGGATACCACAG |
| 13 | SERPINE1 | up | ACTAAGGAAAATAATATTATTTAAACTCGCTCCTAGTGTTTCTTTGTGGTCTGTGTCACC |
| 14 | TIMP2 | up | GTTGAAAGTTGACAAGCAGACTGCGCATGTCTCTGATGCTTTGTATCATTCTTGAGCAAT |
| 15 | TUBB6 | up | GGATGAAATGGAGTTCACCGAGGCGGAGAGCAACATGAACGACCTGGTATCCGAGTACCA |
| 16 | LAMB3 | up | GCATGCCATTGAAACTAAGAGCTCTCAAGTCAAGGAAGCTGGGCTGGGCAGTATCCCCCG |
| 17 | ANXA1 | up | GGCTCTTTGTGGAGGAAACTAAACATTCCCTTGATGGTCTCAAGCTATGATCAGAAGACT |
| 18 | GBP2 | up | ATACAGGCCAAAGAGGTGCTGAAAAAATATTTGGAGTCCAAGGAGGATGTGGCTGATGCA |
| 19 | PTHLH | up | AGCAGGAAAAGAAAAAACGGCGAACTCGCTCTGCCTGGTTAGACTCTGGAGTGACTGGGA |
| 20 | CLIC3 | up | ACACCGCCGGCAACGACGTTTTCCACAAGTTCTCCGCGTTCATCAAGAACCCGGTGCCCG |
| 21 | GBP1 | up | CAAAGATGCATTTACCTCTGTATCAACTCAGGAAATCTCATAAGCTGGTACCACTCAGGA |
| 22 | LAMA3 | up | CCAGGGTTGTAGCCCTGGATACTATCGGGATCATAAAGGCTTGTATACCGGACGGTGTGT |
| 23 | ABCA3 | up | TTTCTATGATGGATATGAAAAATTCAAGGCAGTATGCACAGAATGGACGAGTGCAGCCCA |
| 24 | CTSE | up | CCGTGGATGGAGAATATGCTGTGGAGTGTGCCAACCTTAACGTCATGCCGGATGTCACCT |
| 25 | CD55 | up | ATCTGGGCACACGTGTTTCACGTTGACAGGTTTGCTTGGGACGCTAGTAACCATGGGCTT |
| 26 | SAMD9 | up | ACCAGTCTTCTGAAGGTCATGCCCACAGAAGTCATCGGACCTTACCAAAGTAGGTTGGAG |
| 27 | HBEGF | up | CCCAGTGGAAAATCGCTTATATACCTATGACCACACAACCATCCTGGCCGTGGTGGCTGT |
| 28 | MFI2 | up | CGACACCAACATCTTCACCGTGTATGGACTGCTGGACAAGGCCCAGGACCTGTTTGGAGA |
| 29 | PPL | up | TTTCCATCCGAGAAGCCTCCTCAGTAGTTACTCTGCTCATGAGACAGATCTGGGCTCCAA |
| 30 | PTPN21 | up | TTACTGAAGCTATGCTGGGCAATTCTGGCAATCATTAAAGTGCATAGATTTCTATCTTAA |
| 31 | RND1 | up | CAGTCGCTCTGAACTCATCTCTTCTACCTTCAAGAAGGAAAAGGCCAAAAGCTGTTCCAT |
| 32 | MICAL2 | up | CACTGTATTTGTGCAGATCCTGGCCAGTACAAAGTCGTTGCTCTTGTCTTATCTTCTCTT |
| 33 | QPCT | up | CATATTCCATTTTTAAGAAGAGGTGTTCCAGTTCTGCATCTGATACCGTCTCCTTTCCCT |
| 34 | TNNC1 | up | CAGCGGCACGGTGGACTTTGATGAGTTCCTGGTCATGATGGTTCGGTGCATGAAGGACGA |
| 35 | RASAL2 | up | TGAGAAACTCCTGAATGAAGAAAGGAACCTTGTCTTTCAGGGCATAAGGCGGCGACTTCC |
| 36 | GRAMD1B | up | GGAATGCTTTGTGAGAAGTGGATTCTCTCCGTGTCCCTGCCCCCCACCCAAACTTGAACT |
| 37 | CAMK2N1 | up | TGTTATTGAAGATGATAGGATTGATGACGTGCTGAAAAATATGACCGACAAGGCACCTCC |
| 38 | THSD4 | up | TATTACATAAGCAGGTGAAAGGTAGAAGGCGAATTATGTGAGTAAATATGGTCTGTTTTC |
| 39 | ARL14 | up | CAGGAAATTAACTGGATTTGTGAAGAGCCACATGAAATCAAGAGGAGACACTTTGGCGTT |
| 40 | ABAT | up | AAGTATGCTTTCTCCTGAAAACTTTAGCATTGGGTGCAAATATTCAGTATGGTTCTCGGA |
| 41 | SEMA3C | up | GTTTCAGAGTCACAGTTCCCTTTATTTCACATAAGCCCAAACTGATAGACAGTAACGGTG |
| 42 | CARD6 | up | ACTAGCTTCTGCTAATAGCCCCAATTTGCTTGAATGGGAAAACTCTCTCATTTGACCCTT |
| 43 | SDCBP2 | up | GTGATCAAGAAGGGGAAGATTGTCTCTCTGGTCAAAGGGAGTTCTGCGGCCTGCAACGGG |
| 44 | TP53INP2 | up | GGGGGAAGAGTTTAAGTTATAGGGCATTTGGCTCAAATTTTAAAAGGCCTTTTGTTTACC |
| 45 | PAG1 | up | GCGATCTCTCACATGATGGGGTTCTTTAGTACATGGTAACAGCCATGTCATCTTACACAC |
| 46 | DUSP18 | up | CTCCTTTCTGTGCACAGCACTTTATTGTTACAAAGTACTCTTCCAAAAAGTTACCCTGTG |
| 47 | TIMP1 | up | CATGGAGAGTGTCTGCGGATACTTCCACAGGTCCCACAACCGCAGCGAGGAGTTTCTCAT |
| 48 | ITGA5 | up | GGACTTCCTGTCCAGCTCCAACCTGCAAAGATCTGTCCTCAGCCTTGCCAGAGATCCAAA |
| 49 | PTRF | up | CTTGGGGAACCTCTCACGTTGCTGTGTCCTGGTGAGCAGCCCGACCAATAAACCTGCTTT |
| 50 | EHD2 | up | CTGGGGACTTTCCTGATTGCCAGAAAATGCAGGAGCTGCTGATGGCGCACGACTTCACCA |
| 51 | VIM | up | CCAGATGCGTGAAATGGAAGAGAACTTTGCCGTTGAAGCTGCTAACTACCAAGACACTAT |
| 52 | AXL | up | TGCAATTTCAAGGTTCTAACCCTATACTGTAGTATTCTTTGGGGTGCCCCTCTCCTTCTT |
| 53 | MFGE8 | up | ATCTTCCCTGGCAACTGGGACAACCACTCCCACAAGAAGAACTTGTTTGAGACGCCCATC |
| 54 | MYL9 | up | TGAGGAAGTGGACGAGATGTACCGGGAGGCACCCATTGATAAGAAAGGCAACTTCAACTA |
| 55 | ARL4C | up | AAACGCAGGAAGTCCCTCAAGCAGAAGAAGAAGCGGTAATGCGCCCGGAGCGACCGGGGA |
| 56 | TGFB1I1 | up | CCATCCTGGATAACTACATCTCGGCGCTCAGCGCGCTCTGGCACCCGGACTGTTTCGTCT |
| 57 | COL8A1 | up | GTGTGGGTTGCTCTATTCAAGAACAACGAGCCCGTGATGTACACGTACGACGAGTACAAA |
| 58 | MMP19 | up | AATGGTGGAGGGAGATGCCTGGGTCCTGTTCTTCCTACATAAAATGCAAGAAAACAGCAT |
| 59 | CYR61 | up | ACACTCCATGAGTGTCTGTGAGAGGCAGCTATCTGCACTCTAAACTGCAAACAGAAATCA |
| 60 | AHNAK2 | up | CTTCAAGAAGAAACAATCACGTTTTTTGATGCCCGAGAAAGTTTCTCCCCTGAAGAGAAG |
| 61 | HEG1 | up | AGGATGAGCGTACCACTGAAGTCTGAAGATGTCGCCATTGAACGGACAGTGTTTTCATAT |
| 62 | GABARAPL1 | up | GATCTCTTACCTTTGGAAAATAGGGGTTAGGCATGAAGGTGGTTGTGATTAAGAAGATGG |
| 63 | FAM129A | up | AAAGGTCCAAGGGAATTTAATCTGGAAGAGAACATATGCCAATTTTTAAACTATGACAGC |
| 64 | NCF2 | up | TTACTGTAATTGGCTCTTAAGGCTTGAAGTAACCTTATAGGTTACTCATAAGGCATATAC |
| 65 | EFEMP1 | up | AGGCAGCCATCATAACCATTGAATAGCATGCAAGGGTAAGAATGAGTTTTTAACTGCTTT |
| 66 | CD68 | up | GGGTACCCTTATTTCCTCGACACGCAACTGGCTCAAAGACAATGTTATTTTCCTTCCCTT |
| 67 | PLAUR | up | ACAGGACCCTGAGCTATCGGACTGGCTTGAAGATCACCAGCCTTACCGAGGTTGTGTGTG |
| 68 | LTBP1 | up | CATATGTAAAATTCAATGGAAGAGAGGTGGAACAGTGCTGTTATTTTAAACAGAAGGTTG |
| 69 | GNG11 | up | AAGGAGACTTTCTTAAGCACCATATAGATAGGGTTATGTATAAAAGCATATGTGCTACTC |
| 70 | COL16A1 | up | TGGATGAAAGACTCCGTTGGGAATAAATGGCCAAAGCTTATAGGACTCTGTGACAGGTTG |
| 71 | CAV1 | up | ATTCCTCCTGCTCATATTGTGATTCTCACTTTGGGGACTTTTCTTAAACCTTCAGTTATG |
| 72 | KIAA0247 | up | CCCTGGCTTCTTCAGGTTATCGCACCACTATGGAATCCTTTGCAGAATGGTACTCATATA |
| 73 | ANGPTL4 | up | TGTAGGTCCCCTGGGGACACAAGCAGGCGCCAATGGTATCTGGGCGGAGCTCACAGAGTT |
| 74 | PDGFB | up | AGACTGTGGTAGGGGCAGGGAGGCAACACTGCTGTCCACATGACCTCCATTTCCCAAAGT |
| 75 | MT2A | up | CAACCCTGACCGTGACCGTTTGCTATATTCCTTTTTCTATGAAATAATGTGAATGATAAT |
| 76 | MCAM | up | CACTGAAGTGAGGACACACCGGAGCCAGGCGCCTGCTCATGTTGAAGTGCGCTGTTCACA |
| 77 | FGF1 | up | AATAGTTATGCCTGTACTAAGGAGCATGATTTTAAGAGGCTTTGGCCCAACTGCCTCTTG |
| 78 | LAMC2 | up | ACAGTGGTGACATAGTCTCTGCCCTCATAGAGTTGATTGTCTAGTGAGGAAGACAAGCAT |
| 79 | GEM | up | GGACCTTGCTGGAAACAAAGGCTTAGCAAACAATTTTTGTTCAATGCCCACCAAGACATA |
| 80 | HSPB8 | up | ATGGATACGTGGAGGTGTCTGGCAAACATGAAGAGAAACAGCAAGAAGGTGGCATTGTTT |
| 81 | CD59 | up | ACTGAACTGGGAAACTCAAGGTTCTTTTTACTGTGGGGTAGTGAGCTGCCTTTCTGTGAT |
| 82 | FLNC | up | AATGTCACCTACACTGTCAAGGAGAAAGGGGACTACATCCTCATTGTCAAGTGGGGTGAC |
| 83 | SUSD5 | up | CTTATTCCCCATAGTTGGGTAAAATAAAGCCTGAAGGTACCAACTAGAATACCTGTGCAT |
| 84 | ZBED2 | up | AGACCACCAGTATGAATAAAAGCTTGTTCTGTGTGACCCAGCAAGTGGAAGGACAAAGAA |
| 85 | CD82 | up | CATCAGGGTTCTCTTAGCAACTCAGAGAAAAATGCTCCCCACAGCGTCCCTGGCGCAGGT |
| 86 | CPM | up | GAATGATTCAGTCTTGACGGTGAATGGAAGACACTTACCTAACAAGTACTGCTCATTTAC |
| 87 | STAC | up | CAGCATCTTAGAAGAAATGTAGCCAAATTGGAGTCCATTCTTCTTTAGGGCAGTATATGA |
| 88 | RASGRP3 | up | CATTAAGGCAAAGTAGTTCCAGTGATTTAAAATACGGTTCCAAATACGCTAAAACCAACT |
| 89 | BMP1 | up | CCTGCTCCAGCCTCGATTTGGTTTTATTTTGAGCCCCCATTCCACCACAGTTTCCTGGGG |
| 90 | AOX1 | up | TTGGTTTCCTCTAGGGTGATATTCGTCATTACTCTGTCTCTTCAATCCATCCAGCTAAAT |
| 91 | PBXIP1 | up | AGGCAATCTGATTCTGAAGCTAAAGAGCTTTCATCCTCTTGAGTGTATGTCCCCATAGTG |
| 92 | ARHGDIB | up | TCAGGTTCCAGTAGTTCATTCTAATGCCTAGATTCTTTTGTGGTTGTTGCTGGCCCAATG |
| 93 | QSOX1 | up | TCACCTTCCAGTGTGCAGAAGTTAGAAGGGTCTGGCGGGGGCAGTGCCTTACACATGCTT |
| 94 | PIM1 | up | GCCTGCTGGTTTTATCTGAGTGAAATACTGTACAGGGGAATAAAAGAGATCTTATTTTTT |
| 95 | TRAF1 | up | GAAGAAGAGGGGCATAAACTTTCCTCTTCCTGCCTAGAGGCCCCACCTTTGGTGCTTTCC |
| 96 | AMPD3 | up | AAAGCATTTTTAAAGATTAATCTGAATTAAGCTTTATCAGTGTACTCTTTATCTGTGTTA |
| 97 | RAB3B | up | ACACAGTAGCAAAAGAGAAGATCTCATTTACAAATATCTATGGTGTTTCCTTGTTCTGTG |
| 98 | OPTN | up | ACGTGATGGATTGCATCATTTAAGTGTTGATGTATCACCTCCCCAAAACTGTTGGTAAAT |
| 99 | SERPINB8 | up | AGACTTTTTGGAGAAAAGACGTGTGATTTCCTTCCAGACTTTAAAGAATACTGTCAGAAG |
| 100 | CTSO | up | TGTTTGTGGTATTGCAGATTCCGTTTCTTCTATATTTGTGTGACATGTTGGGCAGATCAA |
| 101 | IL7R | up | CACTACACAGTCTGCAAGATTCTGAAACATTGCTTTGACCACTCTTCCTGAGTTCAGTGG |
| 102 | SOCS3 | up | GCAACTAAACAAACACAAAGTATTCTGTGTCAGGTATTGGGCTGGACAGGGCAGTTGTGT |
| 103 | SMPD1 | up | GTGTACCAAATAGATGGAAACTACTCCGGGAGCTCTCACGTGGTCCTGGACCATGAGACC |
| 104 | HLA-B | up | AAGAGCAGAGATACACATGCCATGTACAGCACGAGGGGCTGCCGAAGCCCCTCACCCTGA |
| 105 | HLA-C | up | AAATTCATGGTGCACTGAGCTGCAACTTCTTACTTCCCTAATGAAGTTAAGAACCTGAAT |
| 106 | NID1 | up | ATACCAAAGATTACTAATTATTCCTCTTTGCCCAAAATACTTGCATCCAAGGTTCTAGTC |
| 107 | LAT2 | up | ACCCCAGCCTGGAGGATCCAGCATCTTCCAGGTACCAGAACTTCAGCAAAGGAAGCAGAC |
| 108 | ITGA3 | up | CGGGATCCTCCACAGAGAGGAGGGGACCAATTCTGGACAGACAGATGTTGGGAGGATACA |
| 109 | IL11 | up | ACGGAGGGGAAAGGGAAGCCTGGGTTTTTGTACAAAAATGTGAGAAACCTTTGTGAGACA |
| 110 | SELL | up | CCAATATGTCAAAAATTGGACAAAAGTTTCTCAATGATTAAGGAGGGTGATTATAACCCC |
| 111 | NAV3 | up | CAGTTTTCAGAGTGTCACAAAGTCAATAGGTCCTTACACGGTGCTATTGCCCTAAGGGAA |
| 112 | ULBP2 | up | TCAATGGGAGACTGTATAGGATGGCTTGAGGACTTCTTGATGGGCATGGACAGCACCCTG |
| 113 | CDKN1A | up | CATCCCTCCCCAGTTCATTGCACTTTGATTAGCAGCGGAACAAGGAGTCAGACATTTTAA |
| 114 | PTPN14 | up | AGCAGAGGATGTTCATGATCCAGACTATCGCTCAGTACAAGTTTGTCTACCAAGTCCTCA |
| 115 | IDS | up | CAAGGTGGAGATCTTTTCCAGTTGTTGATGCCTTGAGTTTTGCCAACCATGGATGGCAAA |
| 116 | MVP | up | ATATCCCCCCAGTCTGCTCAGGCCCCTCAAGCTCCTGGAGACAACCACGTGGTGCCTGTA |
| 117 | S100A3 | up | GGTCTTCTGCGATCAGTTAACCCATTTTACCTAGGAGGCCCAGAGATGTGAGGGCTCCTT |
| 118 | FHL2 | up | TTTACAGCTCTGTAACCTCCCGTTGCGTCAAGTCTAAACCAAGATTATGTGACTTGCAAT |
| 119 | DAB2 | up | ATGTGCTAAACTGGAGGTAACTATTTCTAGGTAGTTGAATTTTTGAAAGTCATGATCAGC |
| 120 | APH1B | up | AAGACAAGAACTTTCTTCTTTACAACCAGCGCTCCAGATAACCTCAGGGAACCAGCACTT |
| 121 | ATP1A2 | up | AGAAACATCTACAGGATCTTTATTGGTGACCTTTTGTAAGACATTAGTTTGAGGTACTAC |
| 122 | AMOTL2 | up | TTTTGTTATGCCACCCTGTACCAGGATTGCTGCCGCATTCCACTGGGTATAACAGTATTT |
| 123 | SYNPO | up | CTCGCCCGCCAAGCCCAGCTCCTTGGACCTGGTGCCCAACCTGCCCAAGGGGGCTCTCCC |
| 124 | ITGA1 | up | CTTCGGAGTGAAAATGCATCTCTGGTTTTAAGTAGCAGCAATCAAAAAAGAGAGCTTGCT |
| 125 | CPE | up | TCTTGTGCTGACTAACTATAAGCATGATCTTGTTAATGCATTTTTGATGGGAAGAAAAGG |
| 126 | IL18 | up | TTGAATGACCAAGTTCTCTTCATTGACCAAGGAAATCGGCCTCTATTTGAAGATATGACT |
| 127 | ADAMTS7 | up | ACCATCCGCACCCAGTGCTGCCGCTCGTGCTCTCCGCCCAGCCACGGCGCCCCCTCCCGA |
| 128 | LYST | up | GTGCATGTTGTAGCAAACATTTCTGTAAATTATCACAAGCTCTGTTACCTTTATATACGC |
| 129 | CNTNAP1 | up | TAGCCAAAGCCATAAAAAACCTGCAACGTAGAGAAAATAATGCAGATACCCTGACTAGCC |
| 130 | SYNGR3 | up | ACAGGCTGCTAGAACAGCCCAGCCCTGTCAGTGTTGTGATCATGGTCCAGTCTTCGGGTT |
| 131 | DOCK3 | up | ATGCTGTGTATTTGTACAGGAATTTGAGCAAAAAATGTATAGAGTGTGATGTCCAATTGG |
| 132 | PTPRE | up | AGAGTTTACGACTTCAGAGACCACATATGGTGCAAACCCTGGAACAGTATGAATTCTGCT |
| 133 | PDGFA | up | GTTTACCTAATATTACCTGTTTTGTATACCTGAGAGCCTGCTATGTTCTTCTTTTGTTGA |
| 134 | MERTK | up | CATTGACCTAAGATTCAAGCACACATAGCCTGAAATTACAGTACTGTATATCCAGAGCCC |
| 135 | IL1R2 | up | CAAACACAGAACTGGAAAAGCAGATGGTCTGACTGTGCTATGGCCTCATCATCAAGACTT |
| 136 | TUBA1A | up | ACCTGAATAGGTTAATAGGTCAAATTGTGTCCTCCATCACTGCTTCCCTGAGATTTGATG |
| 137 | SVEP1 | up | CCCTGCTTAAATGGTGGAAAATGTGTAAGACCAAACCGATGTCACTGTCTTTCTTCTTGG |
| 138 | DENND3 | up | GGGCACCTTCCCACTTTCTAGCTCTGGAGAGGTTGGATTTTGCTTTTGTAAACACATGAA |
| 139 | LBH | up | CTTGTAAACTGCGTAACAAATCTACTTTGTGTATGTGTCTGTTTATGGGGGTGGTTTATT |
| 140 | C4BPB | up | ACCTGGTAGGAAAGAAGACCCTTTTTTGCAATGCCTCTAAGGAGTGGGATAACACCACTA |
| 141 | RBP1 | up | ATATGATCATCCGCACGCTGAGCACTTTTAGGAACTACATCATGGACTTCCAGGTTGGGA |
| 142 | PTPRH | up | TCTGAGGCAGGAAGGAATTTGGGTCTGGAGTTCTGGCTACTTGAGGACCAAAGGCAGGAA |
| 143 | TGFA | up | TTTTTTAAGCATCCTGACAGGAAATGTTTTCTTCTACATGGAAAGATAGACAGCAGCCAA |
| 144 | VLDLR | up | TCTAAACAAATAATACCCCCGTCGGAATGGTAACCGAGCCAGCAGCTGAAGTCTCTTTTT |
| 145 | NMNAT2 | up | ACACCTTAGGAATAGCATTGTAGTAATGGTGATGAATATGCTCTGCCAAATTCATCCAGT |
| 146 | TINAGL1 | up | GATGGGGAGAGGAGACGCTGCCAGATGGAAGGACGCTCAAATACTGGACTGCGGCCAACT |
| 147 | C1orf116 | up | ATTCAGTGTTGTGCCATTGAGTTCCCATGTGGATCATTCTGAAGGTGATCTCCACAAGAG |
| 148 | MICAL1 | up | CCCAGAACAGCAAAAGAAACTATGGGTAGGACAGCTGCTACAGCTCGTTGACAAGAAAAA |
| 149 | DLG4 | up | CCGTTTAACCCATGACATGCAGTCCCACGACATCCCTGTAGCTGTCCCTCTTCAGATATT |
| 150 | PSORS1C1 | up | AATGTTTCCCTCAAGGACCTTTCTGCCTGGAAGTCTGTTAGCCTTTCAGAAGTAACATGT |
| 151 | KIAA1609 | up | GATCCTCAAGCATCTCTACCGTAAGTCCAAAGAATTTAGGTCCCTATTGTCACTTTGTTT |
| 152 | CEACAM1 | up | ATGCTGAACGTAAACTATAATGCTCTACCACAAGAAAATGGCCTCTCACCTGGGGCCATT |
| 153 | ITGA2 | up | ATGTTGGAATGTTATGGGATGTAAACAATGTAAAGTAAAACACTCTCAGGATTTCACCAG |
| 154 | SNCG | up | TGCGCAAGGAGGACTTGAGGCCATCTGCCCCCCAACAGGAGGGTGTGGCATCCAAAGAGA |
| 155 | PTGER4 | up | ACTGTTTCTGGACCCTTATAAAATCCTGTGCAATAGACACATACATGTCACATTTAGCTG |
| 156 | KIAA1539 | up | AAGAGACCCTTCAGGCTTGAATTAAAGCCCTCACCATGCTCACGCCCAAATGGATTATTT |
| 157 | XDH | up | CACAGATATTGTCATGGATGTTGGCTCCAGTCTAAACCCTGCCATTGATATTGGACAGGT |
| 158 | MN1 | up | CAACACCCACGTTGTTTATTTCAAAGAACTTTTCAGCGAAGGGAGAGGAGCTTTCAGAAA |
| 159 | GPR87 | up | GTACATCCACAAATCCAGCAGGCAATTCATAAGTCAGTCAAGCCGAAAGCGAAAACATAA |
| 160 | TMEM59L | up | CTGAAGAGCCACAGGACAATGACTTCCTCAGTTGCATGTCCCGGCGCTCGGGTCTGCCTC |
| 161 | ANXA3 | up | TGGACATTCGAACAGAGTTCAAGAAGCATTATGGCTATTCCCTATATTCAGCAATTAAAT |
| 162 | C3orf52 | up | ACCAGGAGAAAGAGTTTTGTGCTGTATTGTGAGAGATCTCGCCTCTCAGTTAAATGAGCC |
| 163 | STYK1 | up | AGGCTTAGTAGCTCAGTCTTTAACAAGGGCTAGAAAAGAATGTAATCTGATATGGAAGGA |
| 164 | C2orf54 | up | AAATGCTGTATATTTGGTTTTGAAAAAATGAATGTGCTGGGTATACACAGCAGAAAGGGT |
| 165 | ALS2CL | up | CTGTTGGGCCTCAGTTTCTCTTCCCCACACAGTTTATCTTCCGTCACATTGTGCCGGGTG |
| 166 | CORO2B | up | ATTAGCTAGGATCTACTAGATGCATTATACTCCATACCTGCTTTTCCCATGGCCGCCCTA |
| 167 | FBN2 | up | GTTCCCTTGAAAGGGAACACCTGGCATTCTGTGGTGTTTCGTGCTGTCTTAAATAATGGT |
| 168 | SATB1 | up | AGTTGGAAAAGGATAATACAGACTGCACTAAATGTTTTCCTCTGTTTTACAAACTGCTTG |
| 169 | ACPP | up | GAGAGCAACTCAGATACCAAGCTACAAAAAACTTATCATGTATTCTGCGCATGACACTAC |
| 170 | ITGA10 | up | CTTGGCTTCTTTGCCCATAAGAAAATCCCTGAGGAAGAAAAAAGAGAAGAGAAGTTGGAG |
| 171 | MTMR11 | up | CACCCTATCTCCTCTGTATTTTTGTAGTGGAATTTCTATTTAAGGGGCTCATTAAAGCAT |
| 172 | TRIM36 | up | AAGTAGCAAATATCATGGAGAACTTCAGTGTTTAGCAGGCTGTTAATTTCCCATCTATGA |
| 173 | STARD13 | up | AGGGAGAGAGATTTTCTTTGGTCATCCATAATAGAGATTGATAAGATTTAGCAACTGGTG |
| 174 | MYH15 | up | TAAGGTCAGTGTACAGACCCAAGCATGGTGACAGCTTGAAAATATGACTCCAGGCCAAAA |
| 175 | F2RL1 | up | CCTCAGATGGGAATTGCACAGTAGGATGTGGAACCTGTTTAATGTTATGAGGACGTGTCT |
| 176 | TXK | up | AGAAATATATGAGTGGTACCATAGAAACATTACCAGAAATCAGGCAGAACATCTATTGAG |
| 177 | POU2F3 | up | GAAATACCAAAGTGTGAAATACTGTGCTACCTTCCAGAGTTCCCACATGGGCTGGGATTT |
| 178 | CPA4 | up | CCTTCACTGTGCTGAGAATTTCTAGATACTACAGTTCTTACTCCTCTCTTCCCTTTGTTA |
| 179 | TMC7 | up | ACCTCATTGTAAGCTTACCCAAAATTGATAGATGTCAACCTTTTAACAGAAGAACAGAAG |
| 180 | CACNG4 | up | GAAAGCTGTGTTCCAATGAATCCTACCTCTTGCCCAGTCCCAGGCAGAGTAAGCAGGGCC |
| 181 | COL4A5 | up | AACCTGGCTCCCCAGGATTACCTGGACAGAAAGGCGACAAAGGTGATCCTGGTATTTCAA |
| 182 | FAM65B | up | AAGAGCAGTATAAAGAGTTTCAGGATCTGAACCAAGAAGTCATGAATTTGGATGATATTC |
| 183 | MT1P2 | up | |
| 184 | TRANK1 | up | AGTTTCCCCCACAAACAAATTCAGGTAGAGAAGTTGTAGTCGAAGGGAATGTTGGGACTC |
| 185 | CTHRC1 | up | CAAAGATGCGTTCAAATAGTGCTCTAAGAGTTTTGTTCAGTGGCTCACTTCGGCTAAAAT |
| 186 | CCDC80 | up | CGGAGTACTTCTCCATGCTTCTAGTCGGAAAAGACGGAAATGTCAAATCCTGGTATCCTT |
| 187 | SPOCD1 | up | TGGAGGTGGCTGATAAGAGTAGAGTTTCAAAATCTCTTTAAACCTTCCTAAAGCAATGAT |
| 188 | C20orf194 | up | TGAGGATTGGGAATTTCAAATAATTGCAATACCTGAGTTAGAATTCCTTCTAATGGCATC |
| 189 | ALPK2 | up | GGTCTCAAAGACAGGCCATTCTTTATATACACGTTTAGCATTTTTACCAACCTCACATCA |
| 190 | PRICKLE2 | up | ACCAGTAAGGTCTGTTCTTAAACCTACCTAGTTGATTTTCATATCTTTCCATAAAGTGTC |
| 191 | COL12A1 | up | AGTGCATCAACGGGATACAGTTCCATATTGCCTTAAACCTCCTTGTTTTAGACACACTAA |
| 192 | TMCC3 | up | CACACTCCGTCCCCTCTAGTCTGGAGCTGTTAACAGAATCTGCTAGAAACTAGCTTTATT |
| 193 | DCBLD1 | up | GGAGAAAAAGAAAATAACAGGAATTAGGACCACAGGATCTACACAGTCGAACTTCAACTT |
| 194 | FAM46B | up | TTTCCTTCAGGAGACAAAGCAAGTTTGGGGTGGTAGCCTTAATGCCCAGCACCTTGGATT |
| 195 | SPECC1 | up | ACATTACTCAAGGCAAAATAGGAAGCCATCTGCGTGATGTCACATTCCGAAGAAGTCAGA |
| 196 | KRT80 | up | GTTTCTGCATGATCAGTCTGTATGTACTATCTGGAAAGATAACACATACTCCAGCCACCT |
| 197 | C14orf49 | up | AGAAGACACTTACTTTTTATACAGTGTTGTCTTAGTCTATTTATACTAAATGTGTACTAC |
| 198 | C17orf91 | up | TCATCCCAGCATTGCAATGGAGAAAAATGGGAAGCAATGGTTTGGTTGGGAATTTATTCC |
| 199 | HECW2 | up | CACAGCATATCAGGAAAAAGTACACAGTGAGTTCTGTTTATTTTTTGTAGGCTCATTATG |
| 200 | CDYL2 | up | TTAGATCGGGTGTCAGAGGACGACCAATCTTAGGGAATTTCCAGACCAATGAGCAAAATG |
| 201 | LETM2 | up | TTCAGACCACTTAGAAAAATATAATTGCAAAGTCCCATTTCCTCTGTACCATTGTCACCC |
| 202 | PLXNA4 | up | TAGATTATCATCTTTACCAAGTGCAAGTTCCGACTGGCATCAGCAGCATCCCCTGAGCAG |
| 203 | ANKRD29 | up | TAAACCACGAGAAATATATAGGCTTGTCTCTTTGGTCTTTTATTTTGGCTCTATTGTTGG |
| 204 | LIPH | up | AGCATGAGGCAAGGGAAGGAAAAGCAGAGCTTATTTTTCACCTAAGGTGGAGAAGGATCA |
| 205 | WFDC3 | up | ACCAGTCCTGCCCCCAAAACTGACCATGAACCCCAACTGGACTGTGAGGTCTGATTCCGA |
| 206 | GALNT5 | up | GAGCTAGTGGTGTGCTTATTAATGTGGCTTTGGGTAAATGCATTTCCATTGAAAACACTA |
| 207 | GBP3 | up | AATCCTAAAGCATAAGTTAGTCTTTTCCTGATTCTTAAAGGTCATACTTGAAATCCTGCC |
| 208 | CGNL1 | up | ACCAGCTCTCAGAAATGCACGATGAACTGGACAGTGCAAAGCGATCGGAGGACAGGGAGA |
| 209 | PLEKHG5 | up | CTTCCCAGTCCCACAGGCCACCCCTGGCTTGGGCTGGGTTCTGTGAAGTTACGTATTTAT |
| 210 | GCET2 | up | GAAAAGAGGCAAGATTCCCAAAACGAAAATGAAAGAATGTCATCTACTCCCATCCAGGAC |
| 211 | NTN4 | up | ATGTAGACATATGCTTGGCTAAAGGGGAAATGGACTTTAAATTTTAAAGAGCTCATTTGC |
| 212 | RAET1E | up | TCACCAGTAAATGCTTCAGATATCCACTGGTCTTCTTCTAGTCTACCAGATAGATGGATC |
| 213 | C15orf48 | up | AGTGTGCGGAAATGCTTCTGCTACATTTTTAGGGTTTGTCTACATTTTTTGGGCTCTGGA |
| 214 | PPP1R1C | up | AAATTTGCATACATGTGACTGTTCTAACTTTAATACTGCCAGAGCTTAATCCTTGATGTC |
| 215 | UNC13D | up | TACAGGCACAGAGTCTCCTGGAAAAGGGAGAGGGGACCCTGCCAAAGATGAGGCTCCAGC |
| 216 | BRSK1 | up | GGGCGGGGGAGCTGTTTCTATTTTATTTATTGATTAATTTATTATTTTATTTATTGATCA |
| 217 | FANK1 | up | TTCGGCAAAGGTGTCCTAGAAATGGCCAGAGTTTTTGACAGACAGAGTGTAGTCTCCTTA |
| 218 | IGFN1 | up | ATGCCGAAGCCCATGGTCCCTGGATAGCAAAGGTTCAAGTCCTGGAAGGGGCAGTTCTGT |
| 219 | GPR115 | up | AATGCATCACTAGGCCCAACCAATGGATCTAAATTAATGAATCGTCAAGGATGAAATGCT |
| 220 | ATG9B | up | CTCCTTCGATGCGTGGATTACAATGTTCTCTTTGCCAACCAACCAAGTAACCATACCAGA |
| 221 | B4GALNT3 | up | GCCTACTACCAAGACCGGTTCAGCTTTCAGGAGTACATCAAGATTGACCAGCCTGAGAAG |
| 222 | SUSD1 | up | AATTAGTTGATATACTAATGAGAAAATATACTAGCCTGGCCATGCCAATAAGTTTCCTGC |
| 223 | PCDHB9 | up | GAAGGTGCACTGGACAGAGAGAGCAAAGCTGAGTACAACATCACCATCACCGTCACTGAC |
| 224 | WNT9A | up | GCCCAGCTGCGTGGGGTACAGGCATTGCACACAGTGTGAATGGGTCTACACCTGCATGGG |
| 225 | BEST3 | up | GTTTAACTTGAACTCTTATGCAAAAATCCTAATCGTGTCGCAACACGGGACCAGTTACTG |
| 226 | CSRNP1 | up | CCTGAGCCCTAGACCCATGGGTGGCTAAATCCACTGGACTGTGAAGACTATAATTTATTT |
| 227 | RAET1L | up | TGGAAAGCACAGAACCCAGTACTGAGAGAGGTGGTGGACATACTTACAGAGCAACTGCGT |
| 228 | AKNAD1 | up | GTACAAGAATTTTCCAAAAGAATAAAACAGGACTCTCCTTACCATTTGCAAGACAAGAAG |
| 229 | TMEM217 | up | CAATGACTTTGACATTAAAGAGGTCAGAATCATGCGCTGGTTTGGCTTGGTGTCTCGTAC |
| 230 | NAALADL2 | up | TTAAAGATTGCGTACCTAGGTAAGTCACACTGTATAGATAAAAACCTTCTTCTGTATTCC |
| 231 | CYP27C1 | up | AAAGGCATGTTCCAACTGCAGCTGATGTCCCCAAGGTCCCGCTGGTCAGAGCTCTCCTTA |
| 232 | COL28A1 | up | ACAGCTCTGAAAGTTCTAAAATTGCCCTCTTTGATAAACAGAAAGATTTTGTGGATAGCT |
| 233 | BTBD19 | up | GCAGCACTCCGAAGCCTTGTCAACAACCCGCGATACAGTGATGTTTGCTTCGTGGTTGGT |
| 234 | SLC9A7P1 | up | AACCTCATATCAGGATTCCTGACTTTTATGCTACCTGTGTTTCTTCTAGACTGAAGATTT |
| 235 | GAL | down | TATGCAGAGTCAGCCATTCCTGTTCTCTTTGCCTTGATGTTGTGTTGTTATCATTTAAGA |
| 236 | MED12 | down | AGCAACAGACAGCAGCTTTGGTCCGGCAACTTCAACAACAGCTCTCTAATACCCAGCCAC |
| 237 | MYC | down | CGCGCGCCCATTAATACCCTTCTTTCCTCCACTCTCCCTGGGACTCTTGATCAAAGCGCG |
| 238 | SLC7A2 | down | TCAGCAAAAAGTAGCCTTCATGGTTCCATTCTTACCATTTTTGCCAGCGTTCAGCATCTT |
| 239 | DTX4 | down | GGGTCGGAGAAGGAAAAGCCTCTGGGAGCACATTCTGCTGTCATCACAGTCCTTGGCTTC |
| 240 | PSAT1 | down | GATGCATCAGCTATGAACACATCCTAACCAGGATATACTCTGTTCTTGAACAACATACAA |
| 241 | CGREF1 | down | GTATCAGGTCTTTCTATGCAGCTCAGGGAGACCCTAAGTTAAGGGGCAGATTACCAATAA |
| 242 | RGS16 | down | CTCTGAATGTGCTGGGAACCTCTTGGAGCCTGTCAGGAACTCCTCACTGTTTAAATATTT |
| 243 | ADCK3 | down | CAGAAACATGAACAGATACGATTGTGGGATTTTTATCATCTGTGTAGTAGGTGTGTGTAT |
| 244 | TERT | down | TGGGGGTCCCTGTGGGTCAAATTGGGGGGAGGTGCTGTGGGAGTAAAAAACTGAATATAT |
| 245 | HSD11B2 | down | CCACAGGGAAGCATGTACTGTACTTCCCAATTGCCACATTTTAAATAAAGACAAATTTTT |
| 246 | MUC6 | down | TCCCACCTTGTCGCCTACCACACGGTTCCTGACCAGCTCCCTCACTGCCCATGGAAGCAC |
| 247 | MUC5AC | down | CTGTTCCTACCACCAGCACAATCTCTGTTCCTACCACCAGCACAACTTCTGCTTCTACAA |
| 248 | H19 | down | CGTCCCTTCTGAATTTAATTTGCACTAAGTCATTTGCACTGGTTGGAGTTGTGGAGACGG |
| 249 | TMEM52 | down | CTGTACACAGCACTGTGACCTCCTACAGCTCCGTGCAGTACCCACTGGGCATGCGGTTGC |
| 250 | KLRG2 | down | AGGAGGTACCAAGTTGTTTCACACTTGTGATAATCCACTCCCTCGGTTATCTGTTGCTTT |
| 251 | C11orf92 | down | TGTTTTATACCTTCGGGTATAGGTTTGAGTTTTTTCCCCCTCATGCCAAGTATTTAAAAC |
| 252 | IL9RP3 | down | |

1. A method of typing a sample from an individual suffering from cancer, the method comprising:
determining a level of expression for a set of at least 5 genes that are selected from Table 1 in a relevant sample from the individual, whereby the sample comprises expression products from a cancer cell of the patient;
comparing said determined level of expression of the set of genes to the level of expression of the set of genes in a reference sample; and
typing said sample based on the comparison of the determined levels of expression.

2. The method according to claim 1, whereby the set of genes comprises at least ten genes that are selected from Table 1.

3. The method according to claim 1, whereby the sample comprises RNA expression products.

4. The method according to claim 1, whereby the cancer is selected from breast cancer (e.g., BRCA-1 deficient, stage-III HER2-negative, luminal type, basal type, ERBB2 type, ER/PR positive, HER2 positive, ductal carcinoma, lobular carcinoma), ovarian cancer (e.g., BRCA-1 deficient, epithelial ovarian cancer), lung cancer (e.g., non-small-cell lung cancer or small cell lung cancer, metastatic non-small cell lung cancer), liver cancer (e.g., hepatocellular carcinoma), head and neck cancer (e.g., metastatic squamous cell carcinoma of the head and neck (HNSCC), squamous cell carcinoma, laryngeal cancer, hypopharyngeal cancer, oropharyngeal cancer, and oral cavity cancer), bladder cancer (e.g., transitional cell carcinoma of the bladder), and colorectal cancer (e.g., advanced (non-resectable locally advanced or metastatic) colorectal cancer), cervical cancer (e.g., recurrent and stage IVB), mesothelioma, solid cancers (e.g., advanced solid cancers), renal cell carcinoma (e.g., advanced renal cell carcinoma), stomach cancer, sarcoma, prostate cancer (e.g., hormone refractory prostate cancer), melanoma, thyroid cancer (e.g., papillary thyroid cancer), brain cancer, adenocarcinoma, subependymal giant cell astrocytoma, endometrial cancer, glioma, glioblastoma, and other cancers that have metastasized to the brain, esophageal cancer, neuroblastoma, hematological cancers, and lymphoma.

5. The method according to claim 1, whereby said typing indicates that the cancer has a high risk of being or becoming resistant to anti-cancer treatment when the level of expression of the set of genes in a relevant sample from the individual is altered, when compared to the level of expression of the set of genes in a relevant reference sample not being resistant to anti-cancer treatment.

6. The method according to claim 1, further comprising determining a similarity value between the determined level of expression of the set of genes in said individual and the level of expression of said set of genes in a relevant reference sample or reference population.

7. The method according to claim 6, further comprising classifying said individual as having a high risk of being resistant to anti-cancer treatment if said similarity value is below a first similarity threshold value, and classifying said individual as having a low risk of being resistant to anti-cancer treatment if said similarity value exceeds said first similarity threshold value.

8. The method according to claim 7, whereby said anti-cancer treatment is selected from an alkylating agent such as nitrogen mustard, e.g. cyclophosphamide, mechlorethamine or mustine, uramustine or uracil mustard, melphalan, chlorambucil, ifosfamide; a nitrosourea such as carmustine, lomustine, streptozocin; an alkyl sulfonate such as busulfan, an ethylenime such as thiotepa and analogues thereof, a hydrazine/triazine such as dacarbazine, altretamine, mitozolomide, temozolomide, altretamine, procarbazine, dacarbazine and temozolomide, which are capable of causing DNA damage; an intercalating agent such as a platinum agent like cisplatin, carboplatin, nedaplatin, oxaliplatin and satraplatin; an antibiotic such as an anthracycline such as doxorubicin, daunorubicin, epirubicin and idarubicin; mitomycin-C, dactinomycin, bleomycin, adriamycin, mithramycin; an antimetabolite such as capecitabine and 5-fluorouracil, gemcitabine, a folate analogue such as methotrexate, hydroxyurea, mercaptopurine, thioguanine; a mitostatic agent such as eribulin, ixabepilone, irinotecan, vincristine, mitoxantrone, vinorelbine and a taxane such as paclitaxel and docetaxel; a receptor tyrosine kinase inhibitor such as gefitinib, erlotinib, EKB-569, lapatinib, CI-1033, cetuximab, panitumumab, PKI-166, AEE788, sunitinib, sorafenib, dasatinib, nilotinib, pazopanib, vandetaniv, cediranib, afatinib, motesanib, CUDC-101, and imatinib mesylate; a MEK inhibitor including CKI-27, RO-4987655, RO-5126766, PD-0325901, WX-554, AZD-8330, G-573, RG-7167, SF-2626, GDC-0623, RO-5068760, and AD-GL0001; a B-RAF inhibitor including CEP-32496, vemurafenib, GSK-2118436, ARQ-736, RG-7256, XL-281, DCC-2036, GDC-0879, AZ628, an antibody fragment EphB4/Raf inhibitor; a serine/threonine kinase receptor inhibitor, including an Alk-1 inhibitor such as crizotinib, ASP-3026, LDK378, AF802, and CEP37440, and combinations thereof.

9. The method according to claim 1, further comprising determining a strategy for treatment of the patient.

10. The method according to claim 9, whereby said treatment comprises anti-TGFbeta treatment, in combination with said anti-cancer therapy.

11. A method for assigning treatment to an individual suffering from cancer, comprising
(a) typing a relevant sample from the patient according to the method of claim 1;
(b) classifying said sample as having a high risk of being resistant to anti-cancer treatment or as having a low risk of being resistant to anti-cancer treatment;
(c) assigning anti-TGFbeta treatment to an individual of which the sample is classified as having a high risk of being resistant to anti-cancer treatment.

12. The method of claim 11, whereby said anti-TGFbeta treatment is combined with said anti-cancer treatment.

13. The method of claim 11, whereby said anti-TGFbeta treatment comprises administration of LY2157299.

14. The method of claim 12, whereby said anti-TGFbeta treatment comprises administration of LY2157299.
